(19) United States
(12) Patent Application Publication
(10) Pub. No.: US 2012/0027749 A1
(43) Pub. Date: Feb. 2, 2012

(54) Method

(75) Inventor: Aled Clayton, Cardiff (GB)

(73) Assignee: Oxford Biomedica (UK) Limited, Oxford (GB)

(21) Appl. No.: 13/139,682

(22) PCT Filed: Dec. 15, 2009
(86) PCT No.: PCT/GB2009/002885

§ 371 Date: Sep. 28, 2011

(30) Foreign Application Priority Data
Dec. 15, 2008 (GB) 0822836.3

(51) Int. Cl. A61K 39/395; C40B 30/04; G01N 27/447; A61P 35/00; G01N 33/559; G01N 33/574; C12Q 1/68

(52) U.S. Cl. 424/133.1; 436/501; 506/9; 435/6.12; 435/6.11; 204/456; 204/606; 204/601; 204/451

(57) ABSTRACT

The present invention provides a method for detecting a 5T4-positive cancer in a subject, which comprises the following steps: (i) identifying and/or isolating exosomes in a sample from the subject; and (ii) detecting exosome-associated 5T4.

### METHOD

1. Field of the Invention

The present invention relates to methods for detecting cancer, in particular a 5T4-positive cancer in a subject. The invention also relates to the use of such methods to diagnose and monitor the progression and/or treatment of cancer.

2. Background to the Invention

Prostate cancer (PCa) remains the most prevalent male cancer in the western hemisphere, with projected 186,000 new cases, and 28,000 deaths in the USA expected in 2008 (American Cancer Society, Atlanta, Ga. 2008). Whilst advances are being made in understanding the biology underlying this disease, and in many respects in its treatment, there remains a need for better tools for PCa diagnosis and monitoring.

The present invention relates to a novel biomarker based approach for the detection of cancer, such as prostate or bladder cancer, which provides a non-invasive technique for early disease detection, for identifying patients suitable for a particular therapy, and for monitoring the progression of therapy/disease.

### SUMMARY OF ASPECTS OF THE PRESENT INVENTION

The present inventors have surprisingly found that the cancer-associated biomarker 5T4 is expressed and is detectable in exosomes, such as urinary exosomes, from cancer patients.

The expression of 5T4 appears to be higher in/on exosomes than in/on normal cells.

Thus, in a first aspect, the present invention provides a method for detecting a 5T4-positive cancer in a subject, which comprises the following steps:
(i) identifying exosomes in and/or purifying exosomes from a sample from the subject; and
(ii) detecting exosome-associated 5T4.

The method may involve detection of, for example, a 5T4 peptide, polypeptide or nucleic acid encoding such a peptide or polypeptide.

The 5T4-positive cancer may be a cancer of the genitourinary tract, such as prostate or bladder cancer.

The sample may, for example, be a urine sample, a blood sample, or a sample derivable therefrom, such as a serum sample. Detection of 5T4 in urinary exosomes has the advantage that urinary samples are available non-invasively. The sample may alternatively be from a pleural effusion of the lung, or a tissue sample.

In order to verify and/or quantify the result, the method may also involve the detection of one or more:
(i) biomarkers for the cancer, in addition to 5T4;
(ii) exosomal markers; and/or
(iii) markers for a particular tissue or cell-type.

For prostate cancer detection, for example, the method may involve the detection of one or more prostate markers, such as PSA, PSCA and/or PSMA.

In a second aspect, the present invention provides a method for determining whether a given cancer in a subject is a 5T4-positive cancer, by using the detection method according to the first aspect of the invention, where the detection of 5T4 confirms that the cancer is 5T4-positive.

In a third aspect, the present invention provides a method for treating cancer in a subject, which comprises the following steps:
(i) determining whether the cancer is 5T4-positive by a method according to the second aspect of the invention; and
(ii) administration of a 5T4-based therapeutic to a subject which tests positive from step (i).

In a fourth aspect, the present invention provides a method for determining whether a given subject will be suitable for treatment with a 5T4-based therapeutic which comprises the step of detection of a 5T4-positive cancer in the subject by a method according to the first aspect of the invention.

In a fifth aspect, the present invention provides a method for monitoring the progression of a 5T4-positive cancer, using a detection method according to the first aspect of the invention, which comprises the step of comparison of the levels of exosome-associated 5T4 in samples taken from a subject at a plurality of time points, wherein an increase indicates worsening, and a reduction indicates amelioration of the 5T4-positive cancer.

The method of the fifth aspect of the invention may be used for monitoring the progression of a 5T4-positive cancer during treatment, in which method samples are taken from the subject at a plurality of time points during treatment.

The cancer treatment may involve administration of a therapeutic based on 5T4 recognition or expression.

In a sixth aspect, the present invention provides a kit for detecting a 5T4-positive cancer in a subject, which comprises:
(i) an exosome detection, collection and/or purification system; and
(ii) a detection system for 5T4.

The detection system may, for example, detect 5T4 peptide, polypeptide or nucleic acid encoding such a peptide or polypeptide. The detection system may also quantify the level of 5T4.

In connection with the present invention, 5T4 may be detected using one or more of the following methods:

(i) binding to an anti-5T4 antibody;
(ii) binding to a T cell receptor specific for a 5T4 peptide presented by an MHC molecule;
(iii) binding to a nucleic acid sequence showing a high degree of identity to the complement of all or part of a nucleic acid sequence encoding 5T4; and
(iv) amplification of a 5T4 nucleic acid using 5T4-specific primers.

### DESCRIPTION OF THE FIGURES

FIG. 1—Purification of Urine-derived Exosomes

Healthy donor urine was subjected to exosome purification, and at each step, 10 μl of sample was kept for electrophoretic analysis (4-20% gradient polyacrylamide gel, silver stained) (A), demonstrating effective removal of the principal non-exosomal protein bands such as that at ˜80 Kd, and significant enrichment of diverse protein species in the final exosome product (A). Parallel gels were run for immuno-blot analyses, using antibodies against typical exosome proteins as indicated (B). Comparing the sucrose cushion method, with a simpler method of Pisitkun et al, where cell culture media (C) or fresh urine (D) were subject to centrifugation at 17,000 g followed by pelletting at 200,000 g. Exosomes (from sucrose method) and the 200,000 g pellet were normalised for protein differences, and 2.5 μg/well analysed by western blot for markers as indicated.

FIG. 2—Quantification of Urine-derived Exosomes, in healthy donors, and Prostate Cancer Patients

The quantity of exosomes present in each preparation was measured using the BCA protein assay. Values were corrected for urine-specimen volume, and are represented as ng Exosomes per ml of urine. Preparations from 10 healthy donors and 10 PCa patients undergoing standard therapy, at ADT₄ (after 4 weeks ADT), ADT₁₂ (after 3 months of ADT), and at RT₂₀ (and after 20-fractions of radiotherapy) are compared. Bars represent mean+SE. *p<0.5 using the Wilcoxon matched pairs test are shown.

FIG. 3—Characterising Exosomes produced by LNCaP-prostate cancer cell line. Prostate cancer cell lines (LNCaP and DU145), as indicated, were maintained in culture as a source of positive-control prostate cancer exosomes (for subsequent analyses). Whole cell lysates (CL) or exosomes (Exo) were analysed by SDS-PAGE (5 μg/well), with a panel of antibodies as indicated.

FIG. 4—Characterising Exosomes from Healthy Donor Urine

Six healthy donors (detailed in Table-3), provided urine specimens and exosomes were purified. Western blots were performed with 5 μg urine-derived exosomes/well, or with 5 μg LNCaP-derived exosomes (Exo) or 5 μg LNCaP whole cell lysates (CL). Blots were probed with antibodies against PSA, TSG101, 5T4, CD9 and GAPDH, as indicated

FIG. 5—Characterising Exosomes from PCa patients

Urinary exosomes (5 μg/well), isolated from 8 PCa patients (at ADT₄, ADT₁₂ or RT₂₀), were subject to western blot analyses with a panel of antibodies as indicated. Whole cell lysates (CL) or exosomes (Exo) of LNCaP (5 μg/well) was included on each gel as positive controls

FIG. 6—Summary of patients' Western blot data

FIG. 7—Characterisation of HT1376-derived exosomes using Western blotting, flow cytometry and electron microscopy

Cell (CL) or exosome (Exo) lysates (5 μg/well) were compared by western blotting using a range of antibodies as indicated. This demonstrated relative enrichment of several proteins in exosomes. Some markers, such as gp96, were absent from exosomes which indicated negligible contamination of the preparations by cellular debris (this is representative of 3 experiments) (A). Exosomes coupled to latex-beads were analysed by flow cytometry and this revealed positive expression of tetraspanin molecules on the exosome surface. Median fluorescence intensity values (MFI) are shown (representative of >5 experiments) (B). Intentional contamination of purified exosomes with increasing amounts of FBS prior to coupling to latex beads reveals a decrease in signal intensity for CD9 (mean±SEM, n=6, **p<0.001, 1-way ANOVA with Tukey's post test) (B line graph). Material pelleted at 70,000 g from cell conditioned medium was overlaid on a linear sucrose gradient (0.2 to 2.02M) and ultracentrifuged for 18 h at 210,000 g. Collected fractions were analysed by refractometry to ascertain fraction density and thereafter by western blot using antibodies to TSG101 which is an exosome marker. TSG101 floats at typical exosome densities of between 1.1 and 1.2 g/ml (Representative of 4 experiments) (C). Transmission electron micrograph of a typical exosome preparation revealing heterogeneous vesicles between 30 and 100 nm in diameter (D).

FIG. 8—Summary of over-representation analysis of the nano-LC/MS-derived protein identifications against gene sets from ExoCarta and GeneGO

To facilitate comparison with Exocarta gene sets our protein list was first converted to an EntrezGene identified gene list, before undertaking ORA using the hypergeometric distribution. Results were filtered to include comparisons with MS-based studies only, and to those reporting 10 or more matching genes. This demonstrates how well our MS data compare with exosome protein profiles from specified cell types, displayed as the −log(p-value), corrected for false detection rate (A). ORA analysis using MetaCore utilised the SwissProt IDs for the identified protein list. For clarity, we report the top ten over-represented genes contained within each of the group headings of Disease Biomarker (B), Diseases (C), Biological Process (D) and Cellular Compartment (E). The dotted line indicates p=0.05, hence columns to the left of this are not statistically significant.

FIG. 9—Validation of some MS-identified proteins by western blot and flow cytometric analysis

HT1376 exosomes (5-20 μg/well), purified by the standard sucrose cushion method, were analysed by western blot for expression of a range of MS identified proteins as indicated (A). The 70,000 g pellet, obtained from HT1376 cell-conditioned medium, was subject to fractionation by centrifugation on a linear sucrose gradient (0.2 M to 2.5 M). Fifteen total fractions were collected, and the density measured by refractometry. Thereafter, one third of each fraction was coupled to latex beads followed by flow cytometric analysis for exosomal surface expression, as indicated (B). In parallel, the remainder ⅔^{rd} of each fraction was subjected to western blotting, for proteins as indicated (C). The data reveal proteins floating at a recognised exosomal density range (1.12-1.2 g/ml). (The data are representative of 2 experiments).

FIG. 10—Analysis of HT1376-derived exosomes using 2DE and MS

Protein extracts from HT1376 exosomes were resolved by 2DE on a pH 3-10 non-linear gradient. Proteins were visualised by silver staining (A). 32 spots were randomly chosen, gel plugs excised, and peptides recovered following trypsin digestion. Of these, successful identifications were obtained for 17 spots (annotated in A), and the details of the MS identifications listed (B). A representative MS/MS analysis from the data set is shown in (C), the peptide is from integrin alpha-6, spot 10. The peptide has a precursor mass of 1191.9 and is annotated to show the derived peptide sequence.

FIG. 11A and B—Exosomes express surface 5T4, detected by a microplate assay

### DETAILED DESCRIPTION

### 5T4

The present invention relates to a method for detecting a 5T4 positive cancer in a subject.

5T4 is a 72 kDa transmembrane glycoprotein expressed widely in carcinomas, but having a highly restricted expression pattern in normal adult tissues (see Table 1). It appears to be strongly correlated to metastasis in colorectal and gastric cancer. The full nucleic acid sequence of human 5T4 is known (Myers et al., 1994 J Biol Chem 169: 9319-24).

TABLE 1
Distribution of Human 5T4

| | 5T4 Frequency |
|---|---|
| Tumour Type | (%) |
| Breast | 84 |
| Ovarian | 71 |
| Gastric | 74 |
| Colorectal | 85 |

5T4 expression is associated with many cancers, including but not limited to: mesothelioma, renal cancer, prostate cancer, cancer of the breast, ovary, lung, cervix, colorectum, liver, stomach, pancreas, bladder, endometrium, brain and esophagus.

In connection with the present invention, a “5T4-positive” cancer is a cancer which is associated with 5T4 expression: a cancer for which 5T4 is a tumour-associated antigen.

Overexpression of 5T4 is particularly associated with cancers of high metastatic potential and poorer prognosis, so the detection method of the present invention can also be used as a prognostic indicator.

### Exosomes

Exosomes are nanometer-sized (40-100 nm) vesicles found in some body fluids, such as serum and urine. Exosomes originate as internal vesicles of multi-vesicular bodies (MVBs) in cells. They were first described as products of circulating blood cells, such as erythrocytes and lymphocytes. In the kidney, exosomes are released into the urine by fusion of the outer membrane of the MVBs with the apical plasma membrane.

Proteomic analysis of urinary exosomes using tandem mass spectrometry revealed membrane proteins from each cell type facing the urinary space. In addition, the lumens of exosomes contain cytosolic proteins and mRNA from their cells-of-origin that are entrained when exosomes are formed in the MVBs.

Exosomes can be detected and/or purified on the basis of their expression of exosomal markers such as tumour susceptibility gene (TSG101), aqua-porin-2 (AQP2), neuron-specific enolase (NES), annexin V, podocalyxin (PODXL) and CD9.

### Exosome Isolation

Exosomes may be isolated or purified by methods known in the art such as ultracentrifugation (Pisitkun et al (2004) PNAS 101:13368-13373) or ultrafiltration (Cheruvanky et al (2007) Am. J. Physiol. Renal Physiol. 292:F1657-F1661). Methods are also known involving continuous flow electrophoresis and chromatography procedures which may precede centrifugation (Taylor and Gercel-Taylor (2005) Br J Cancer 92:305-311). Cross-flow ultrafiltration may also be used as part of the exosome purification method (Lamparski et al (2002) J Immunol. Methods 270:211-226).

It is also possible to perform the method of the invention by identifying exosomes (and thus detecting exosome-associated 5T4) without isolating or purifying the exosomes. For example, exosomes may be captured on to a microplate, for example by immunoaffinity capture, followed by 5T4 detection.

It is also possible to detect exosome-associated 5T4 in the fluid-phase, for example by using a bifunctional molecule, capable of detecting both exosomes and 5T4 or bi-colour immunofluorescence using antibodies to exosomes and/or 5T4.

### 5T4 Detection Methods

The detection method of the present invention may involve detection of a 5T4 peptide, polypeptide, or nucleic acid encoding such a polypeptide.

As used herein, the term “polypeptide” refers to a polymer in which the monomers are amino acids and are joined together through peptide or disulphide bonds. “Polypeptide” refers to a full-length naturally-occurring amino acid chain or a fragment thereof, such as a selected region of the polypeptide that is of interest in a binding interaction. A polypeptide which comprises a fragment of 5T4 may be at least 100, 200, 300 or 400 amino acids in length. Full length human 5T4 is 420 amino acids in length

“Peptide” thus refers to an amino acid sequence that is a portion or fragment of a full-length 5T4, which may be between about 8 and about 100 amino acids in length.

The peptide may be or comprise a T-cell epitope of 5T4.

A T cell epitope is a short peptide derivable from a protein antigen. Antigen presenting cells can internalise antigen and process it into short fragments which are capable of binding MHC molecules. The specificity of peptide binding to the MHC depends on specific interactions between the peptide and the peptide-binding groove of the particular MHC molecule.

Peptides which bind to MHC class 1 molecules (and are recognised by CD8+ T cells) are usually between 6 and 15, for example between 8 and 12 amino acids in length. The amino-terminal amine group of the peptide makes contact with an invariant site at one end of the peptide groove, and the carboxylate group at the carboxy terminus binds to an invariant site at the other end of the groove. The peptide lies in an extended confirmation along the groove with further contacts between main-chain atoms and conserved amino acid side chains that line the groove. Variations in peptide length are accommodated by a kinking in the peptide backbone, often at proline or glycine residues.

WO 03/068816 describes various MHC class I epitopes of 5T4, including PLADLSPFA, LHLEDNALKV, LEDNALKVLH, HLEDNALKV, LEDNELKVL AND LADNALKV.

Peptides which bind to MHC class II molecules are usually at least 10 amino acids, for example between 10 and 50, 10 and 30 or 15 and 25 amino acids in length. These peptides lie in an extended confirmation along the MHC II peptide-binding groove which is open at both ends. The peptide is held in place mainly by main-chain atom contacts with conserved residues that line the peptide-binding groove.

WO 03/068815 describes various MHC class II epitopes of 5T4, including YRYEINADPRLTNLSSNSSDV and QTSYVFLGIVLALIGAIFLL. WO 2006/120473 and WO2008/059252 describe further peptide epitopes of 5T4.

Human 5T4 is as characterised by Myers et al (as above), the sequence of which appears in GenBank at accession no. Z29083. For veterinary applications, the relevant homologue is also considered. The sequences of canine and feline 5T4 are described in WO 01/36486 and WO 02/38612.

The 5T4 peptide detectable using the method of the invention may comprise an antibody binding site. Myers et al (as above) describes a mouse polyclonal anti-5T4 antiserum which binds the 5T4 core protein.

5T4 polypeptides or peptides may be detected using any of the methods known in the art including ELISA, western blot, FACS analysis, immunoprecipitation, in situ hybridisation and mass spectrometry.

Anti-5T4 antibodies are known in the art. Myers et al ((1994) J. Biol. Chem. 269:9319-9324) describes a mouse polyclonal anti-5T4 antiserum. More recently the anti-5T4 antibody H8 has been described (Boghaert et al (2008) Int. J. Oncol. 32:221-234).

The term “antibody” in connection with 5T4 detection include functional antibody fragments, such as Fab, F(ab)₂, Fv, scFv and domain antibodies (dAbs) together with fusions and mimetics thereof such as Affibodies, DARPins, Anticalins, Avimers, and Versabodies.

WO 03/020763 and WO 99/18129 outline the design and production of soluble T-cell receptor formats that would be suitable for the detection of 5T4.

As used herein the term “nucleic acid” refers to a nucleotide sequence, which may be DNA or RNA, single stranded or double stranded, which is capable of, or is complementary to a sequence which is capable of encoding a 5T4 peptide or polypeptide.

Previously described Northern analysis has demonstrated that a 2.5 kb mRNA is associated with 5T4 expression (Myers et al (as above).

A nucleic acid sequence encoding a 5T4 polypeptide, may be or comprise a 1260 base section, capable of encoding the full-length protein. A nucleic acid sequence encoding a fragment of 5T4 may be between 300 and 1200 bases, for example between 500 and 1000 bases. A nucleic acid sequence encoding a peptide of 5T4 may be between 24 and 500 bases, for example between 50 and 300 bases in length. Primers for amplification of 5T4 nucleotide sequences have previously been described (Myers et al (1994) as above).

Probes for detecting 5T4 nucleotide sequences may be used which show a high degree of homology to the complement of the sequence. A suitable probe may be a single-stranded DNA or RNA that has a sequence of nucleotides that includes between 10 and 50, for example between 15 and 30 and or at least about 20 contiguous bases that are the same as (or the complement of) an equivalent or greater number of contiguous bases of a 5T4-encoding sequence. The nucleic acid sequences selected as probes should be of sufficient length and sufficiently unambiguous so that false positive results are minimised. To minimise false positives, the probe may be based on portion of sequence which is largely unique to 5T4, i.e. which does not show a high degree of identity to any other sequences.

Nucleic acid probes may be labelled for ready detection upon hybridisation. For example, the probe may be radiolabled. The preferred method of labelling a DNA fragment is by incorporating α32P dATP with the Klenow fragment of DNA polymerase in a random priming reaction, as is well known in the art. Oligonucleotides are usually end-labelled with γ32P-labelled ATP and polynucleotide kinase. However, other methods (e.g. non-radioactive) may also be used to label the fragment or oligonucleotide, including e.g. enzyme labelling, fluorescent labelling with suitable fluorophores and biotinylation.

5T4 nucleic acid may be detected using any of the method known in the art, including northern blot, polymerase chain reaction (PCR) and quantitative PCR. WO 02/18645 describes various methods for the detection of 5T4 RNA, including in vitro amplification by reverse transcriptase PCR, ligase chain reaction, DNA signal amplification, amplifiable RNA reporters, Q-bate replication, transcription-based amplification, isothermal nucleic acid sequence based amplification, self-sustained sequence replication assays, boomerand DNA amplification, strand displacement activation, cycling probe technology; followed by detection by methods such as gel electrophoresis, capillary electrophoresis, conventional enzyme-linked immunoabsorbent assay (ELISA), nucleic acid hybridisation using specific labelled probes, Southern blot analysis, Northern blot analysis, electrochemiluminescence, laser-induced fluorescence, reverse dot-blot detection and high-performance liquid chromatography.

Using western blot, exosomal 5T4-expression has not been detected in healthy individuals. Other detection methods may be able to detect trace amounts of 5T4. Expression of exosomal 5T4 may be 10, 50, 100, 500 or 1000-fold greater in an individual with a 5T4-associated cancer than in a healthy individual.

The method of the present invention may involve the step of comparing exosomal 5T4 in the subject with an equivalent sample taken from a healthy individual. This may involve normalisation of the value obtained with the number of exosomes in the sample.

In addition to 5T4, the detection method may involve the detection of one or more other biomarkers.

These further biomarkers may be characteristic of, for example, cancer (or a particular type thereof), exosomes, or a particular tissue or cell type.

### Therapeutics Based on 5T4 Expression or Recognition

Various therapeutics based on 5T4 recognition have been described, such as the use of a conjugate of calicheamycin and an anti-5T4 antibody (Boghaert et al (2008) as above) and a fusion of a Fab recognising 5T4 and the superantigen Staphylococcal enterotoxin A (Shaw et al (2007) Br. J. Cancer 26:567-574).

It is also possible to stimulate an immune response useful in cancer therapy by inducing and/or increasing 5T4 expression in a subject.

### Subject

The subject from which the sample is taken in the method of the first aspect of the invention may be a mammalian subject, such as a human.

The subject may be a non-pregnant mammal. During pregnancy, placental exosomes may be found in the maternal circulation, which may express 5T4 (Taylor et al (2006) J. Immunol. 176:1534-1542). In order to avoid complication of the tumour detection method, for pregnant mammals, it may therefore be necessary to remove placental exosomes from the detection system. Such removal may be either physical, or notional, whereby placental exosomes are discounted from the detection process, for example by negative selection using a placental marker or positive selection using a marker associated with another tissue, such as a tissue which has the potential to be associated with a 5T4-based cancer.

### Kits

The sixth aspect of the invention provides a kit for detecting a 5T4-positive cancer in a subject, which comprises:
(i) an exosome detection, collection and/or purification system; and
(ii) a detection system for 5T4.

The exosome detection, collection and/or purification system may be suitable for use with the known untracentrifugation or ultrafiltration systems mentioned above. Alternatively, the exosome detection system may be a substrate suitable for exosomal capture, such a microtitre plate coated with an antibody against an exosomal marker.

The detection system may be suitable for detection of a 5T4 peptide, polypeptide or nucleic acid by any one of the methods mentioned above. The detection system may comprise an anti-5T4 antibody.

The kit may also comprise instruction for using the exosome detection, collection or purification system and/or the 5T4 detection system.

The invention will now be further described by way of Examples, which are meant to serve to assist one of ordinary skill in the art in carrying out the invention and are not intended in any way to limit the scope of the invention.

### EXAMPLES

### Example 1

### Purification of Urinary Exosomes

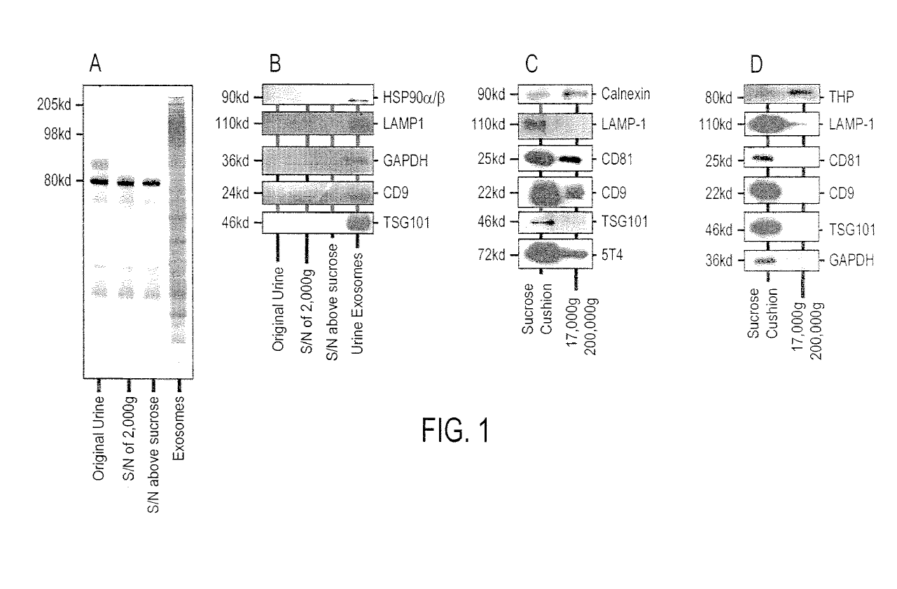

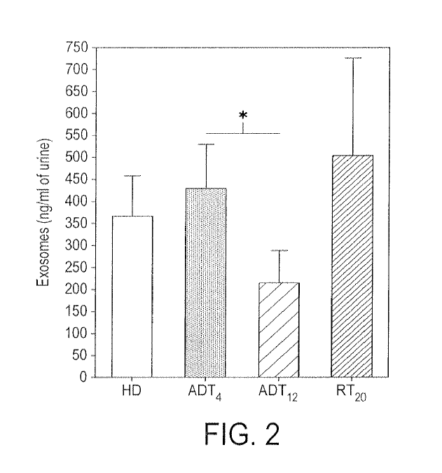

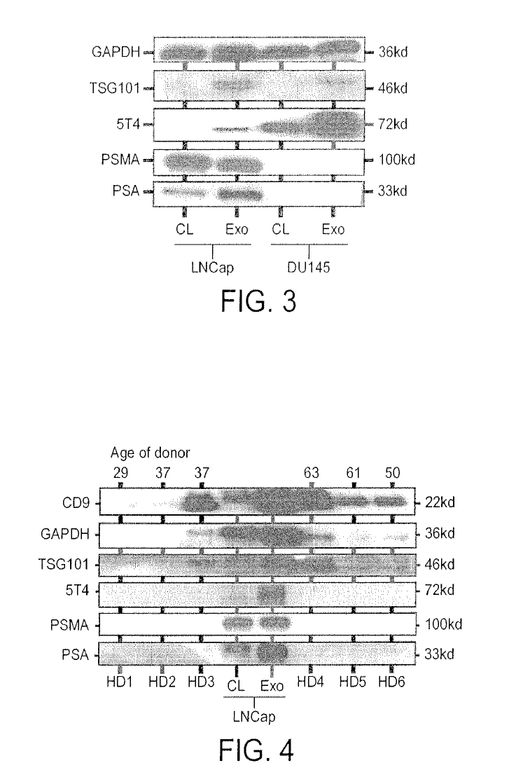

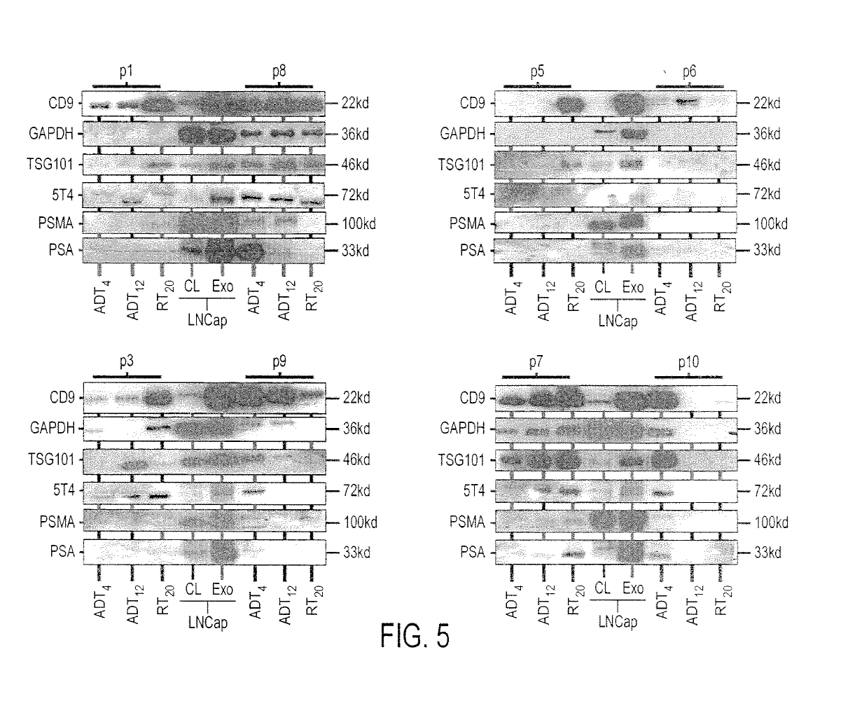

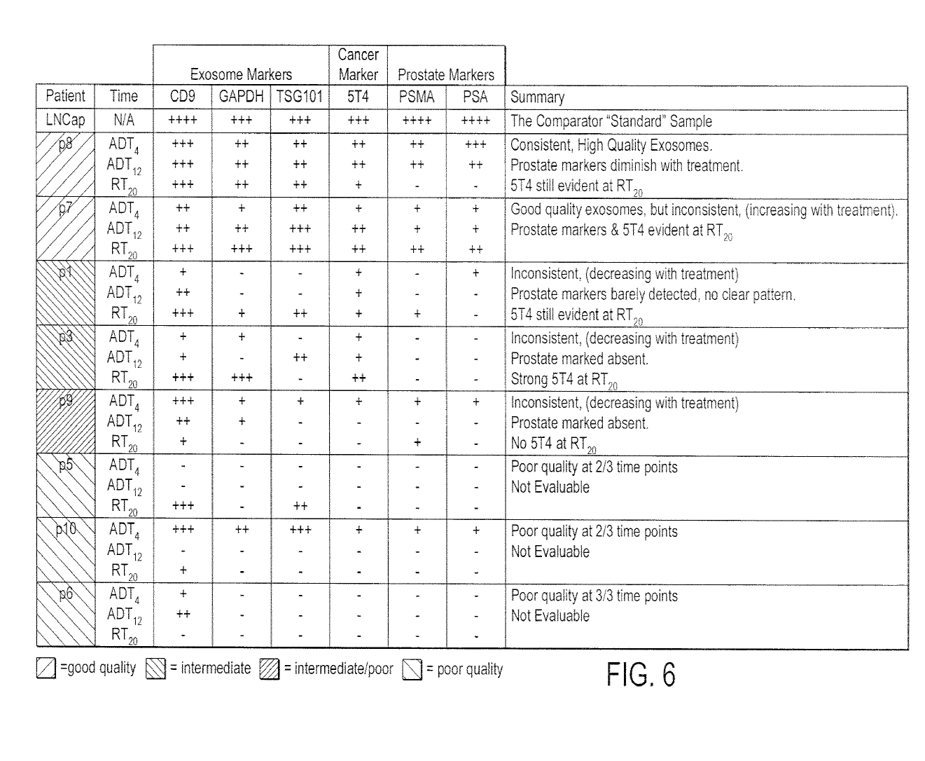

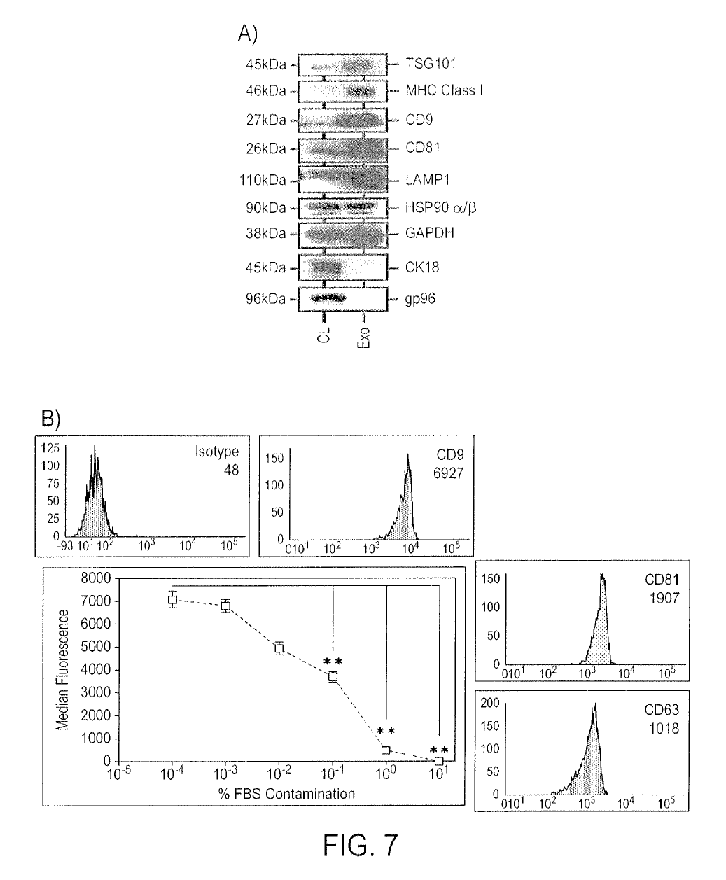

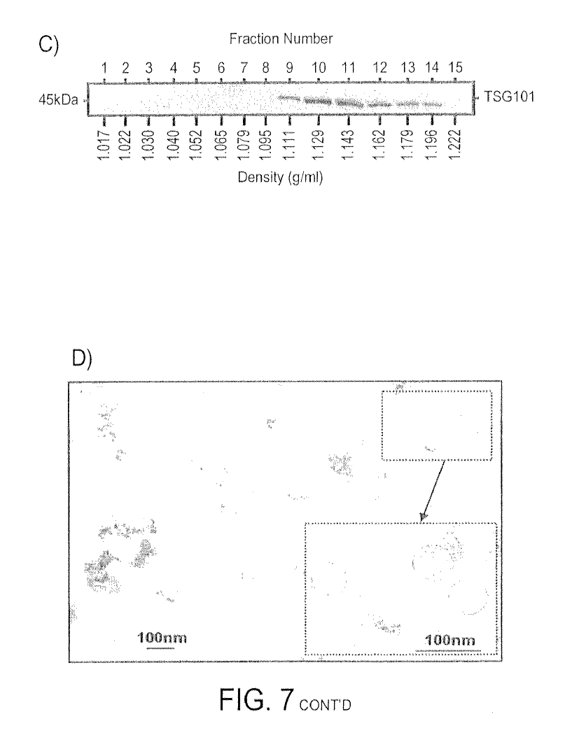

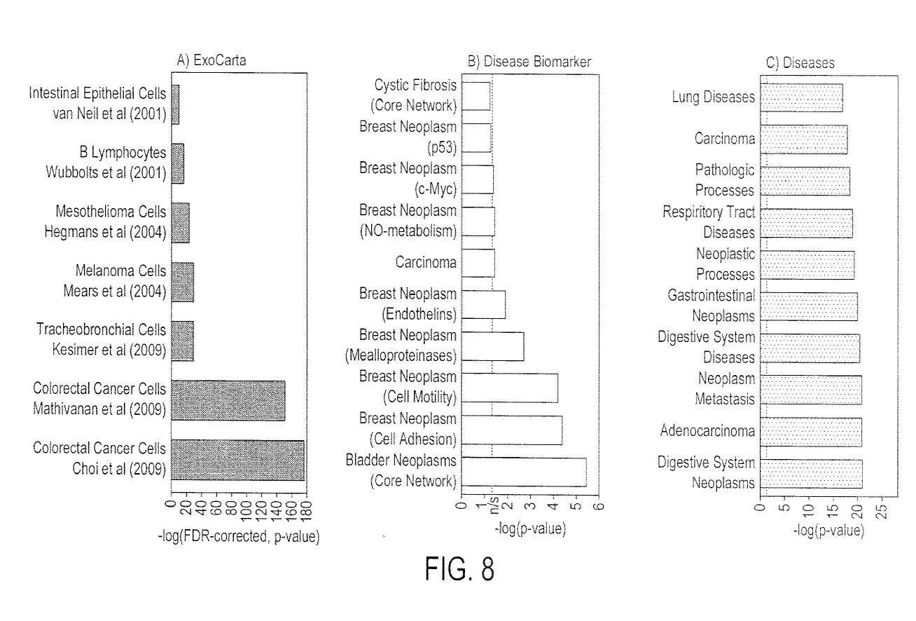

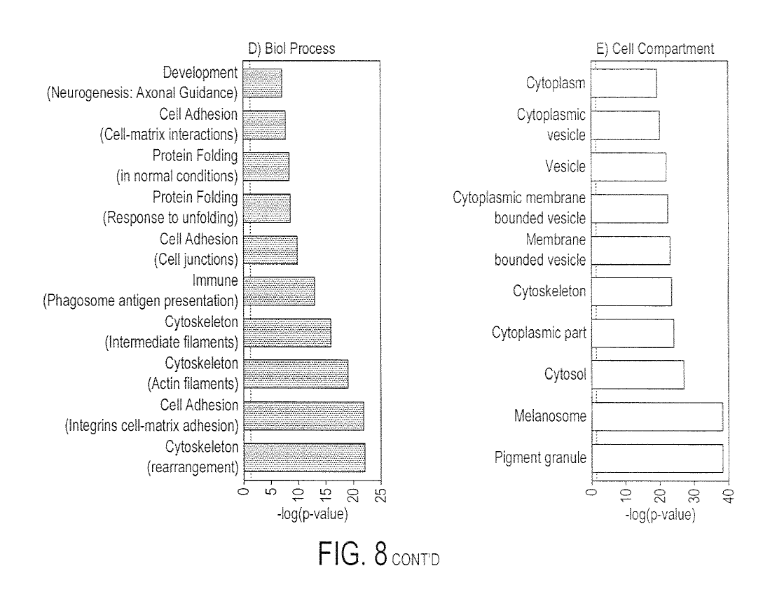

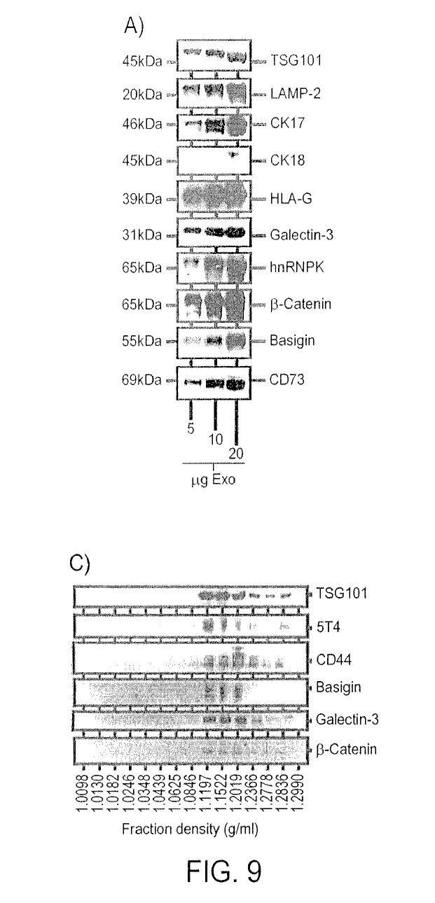

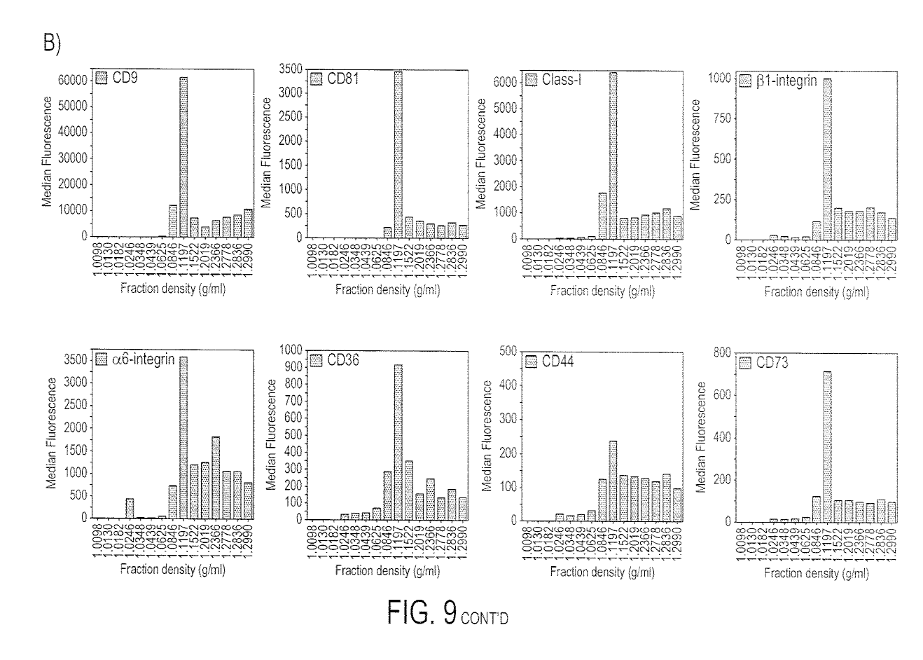

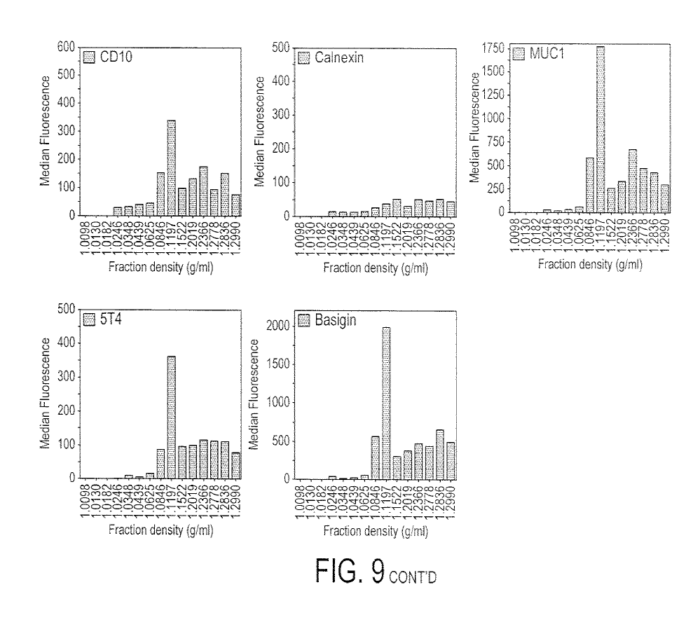

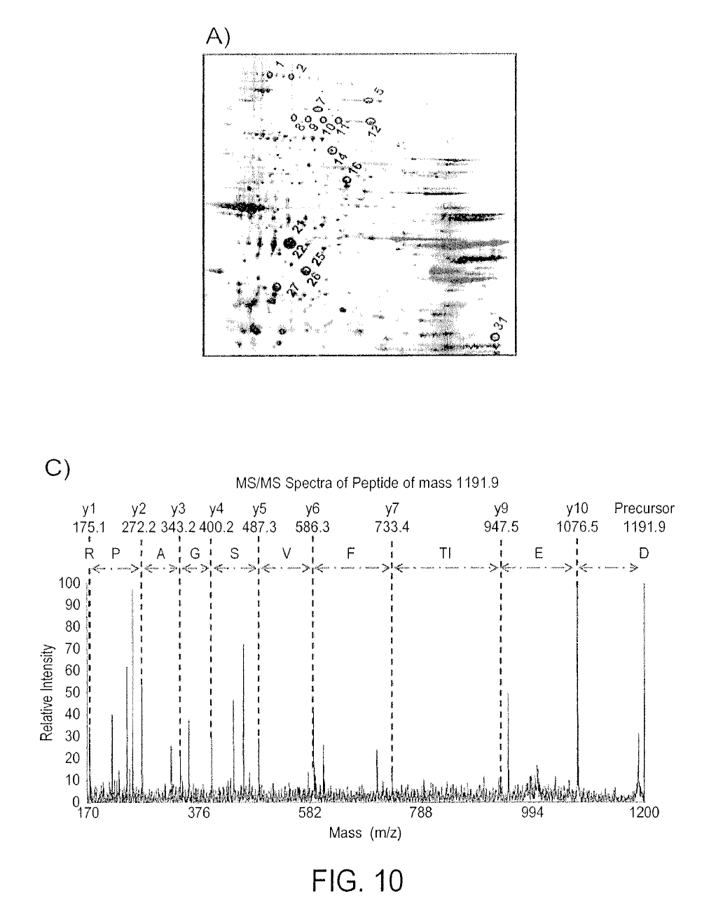

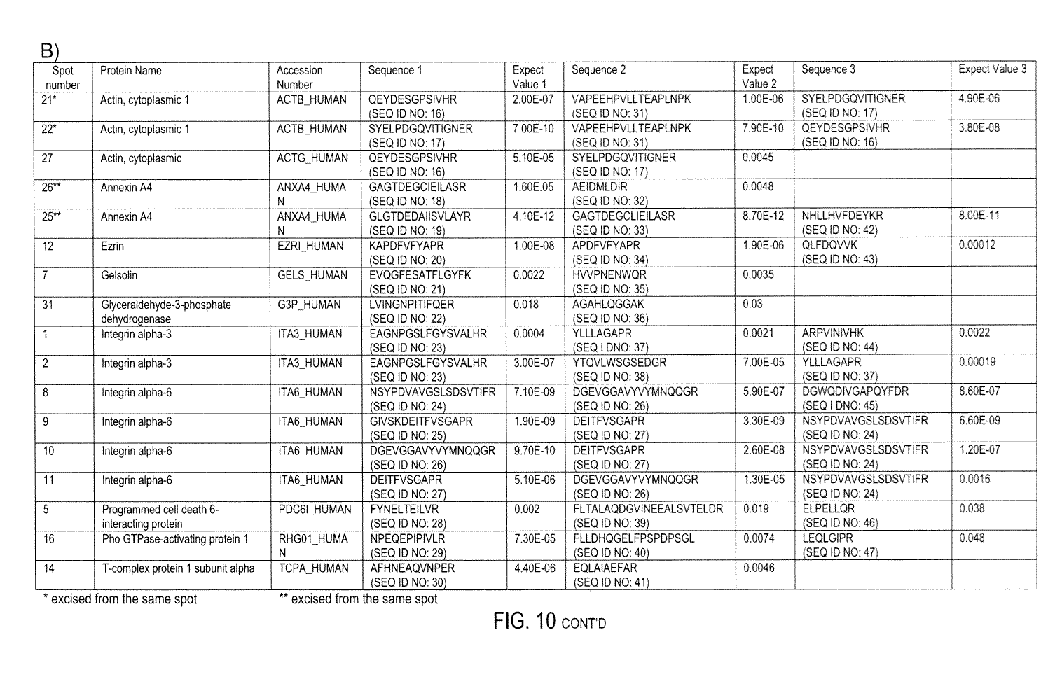

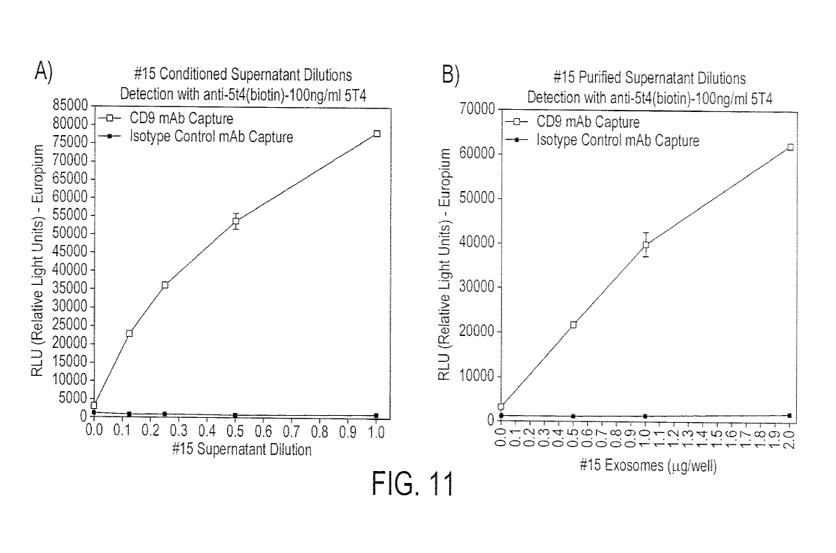

A standardised method was used, designed for exosome-purification from cell culture supernatant, and applied to fresh-urine as an exosome source. With this method, exosomes are isolated based on their buoyancy characteristics (Raposo et al (1996) J. Exp. Med. 183:1161-1172). Analysis of protein content of urine at multiple steps throughout purification, revealed the method was effective in eliminating principal non-exosomal contaminants (FIG. 1a), (such as the band at 80 Kd) while significantly concentrating vesicles bearing a distinct protein repertoire, across the entire molecular weight spectrum (FIG. 1a). Performing immuno-blot analyses on parallel gels revealed typical exosomal proteins were only detected in the final exosome-product (FIG. 1b).

This method was also directly compared with the method of Pisitkun et al ((2004) PNAS 101:13369-13373), using cell culture supernatants (FIG. 1c) or healthy donor urine (FIG. 1d) as source material. This latter method, involves pelletting debris at 17,000 g, followed by a 200,000 g spin of the supernatant, to pellet exosomes. Our sucrose method results in a pellet which is more enriched in exosomes, evident by strong band intensity for exosome markers such as CD9, TSG101 and LAMP-1. Importantly, the sucrose method resulted in good enrichment of tumour associated antigens; in this case 5T4 (FIG. 1c), indicating an important advantage in analysis of exosomes over pelleted sediment. Although many markers were detected in the comparator preparation, these were at a lower level. The more intense band for calnexin (a non-exosomally expressed marker), is a direct demonstrator of more non-exosomal contaminants when using the comparator method (FIG. 1c). A similar advantage of using the sucrose-cushion method was apparent using fresh urine as the source material (FIG. 1d), showing higher levels of exosome expressed proteins, and reduced contamination with Tamm Horfsall protein (THP). The data support this approach for enriching exosomes from fresh urine specimens; and confers some advantages over previously published urine-exosome protocols.

### Example 2

### Changes in Urine-Exosome Quantity During PCa Therapy

The quantity of exosomes present in each preparation was measured, corrected for starting urine volume, and values compared across the healthy donor and patient groups are shown in FIG. 2. Prostate cancer patients on average had 1.2-fold higher levels of urinary exosomes (at ADT₄) compared to healthy men (FIG. 2A). There was broad variation in the exosome-content across both the healthy donors (366.8±92.56, n=10 mean±SE) and patients (443.2±109.7, n=10, ADT₄), so this difference did not reach significance. The exosome levels were also measured after three months of androgen deprivation therapy (ADT₁₂) (224.9±82.7, n=10), and after 20-fractions of radiotherapy (RT₂₀) (499.6±225.6, n=9). There was a 2-fold decrease in the average exosome levels following 12 weeks hormone-treatment, with 8 out of 10 patients showing a decrease in urinary exosome quantity. In terms of radiation treatment, there was no significant difference compared to ADT₄ or to ADT₁₂, as 3 out of 9 patients demonstrated a further decrease in exosome levels, whilst 6 out of 9 had increasing urinary exosome levels. Using serum-PSA levels as a surrogate marker for prostate cancer outcome, indicated that in 9/10 patients the standard therapy of ADT and RT was successful in tumour bulk reduction.

In conclusion it is not possible to demonstrate a correlation between locally advanced PCa with the quantity of exosomes present in urine, and there is no correlation between serum PSA and urinary-exosome levels. From the current data set, there is some suggestion however, that at ADT₁₂ there is a decrease in the amount of exosomes present.

### Example 3

### Prostate Cancer Cell Lines Produce Typical Exosomes, Positive for Prostate and Cancer-Associated Antigens

Two well characterised prostate cancer cell lines were maintained in culture, as a source of PCa-exosomes, and the expression of typical exosome-markers (e.g. the tetraspanin CD9) and some known markers of prostate (PSA and PSMA) were examined. The LNCaP ells (whole cell lysates) were directly compared to LNCaP-exosomes by immuno-blot, revealing positive exosomal expression of PSA and PSMA. There was also clear positive exosomal expression of 5T4 by LNCaP-exosomes. Both PSA and 5T4 were particularly enriched in exosomes, compared to the parent cell (FIG. 3A). The DU145 cell line, which does not express PSA or PSMA served as a control demonstrating specific staining. Staining for GAPDH showed equal loading of wells. It was concluded that exosomes isolated from PCa cells express molecules typical of exosomes from other cellular sources together with prostate markers and tumour-associated antigen(s). This simple immuno-blot panel was therefore considered suitable for analysis of urinary exosomes in following studies.

### Example 4

### The Phenotype of Healthy Donor Urinary Exosomes

Similar analyses of urinary-exosomes were performed from healthy donors (HD), and compared expression levels for these molecules to those of LNCaP-derived exosomes. Markers such as TSG101 and CD9 were detected in most HD-specimens by western blot, albeit at low levels compared to the LNCaP standard. Prostate markers (PSA and PSMA) were not expressed in any healthy donor specimens, regardless of the age of the donor, indicating that few if any exosomes in healthy donor urine arise from the prostate. The tumour antigen 5T4 was not found in any of the HD specimens (FIG. 4).

In conclusion, examining urinary-exosomes obtained from different donors by this method reveals variation in exosome-quality across the samples. In cases where exosome-quality was moderate/good (i.e. comparable to LNCaP exosomes), healthy donor urinary-exosomes could be confirmed negative for PSA, PSMA and 5T4.

### Example 5

### Phenotype of PCa Patient's Urinary Exosomes, and Evaluating Changes with Treatment

PCa patient derived exosomes were examined by western blot in a similar manner. The data from 8 individual patients are shown (FIG. 5). Overall, there was variability in band intensity (with multiple markers) across the sample series, with weak staining in most occasions compared to the LNCaP-exosomes. Although weak, there was evidence of positivity for exosome-markers (such as CD9) in 20 of 24 samples. There was variation across the patient cohort, and variation from within an individual's sample series (ADT₄, ADT₁₂ and RT₂₀). As great attention was paid towards loading 5 μg of sample per well, we believe the results more likely reflect the variable exosomal content of the sample, rather than technical issues of sample loading.

It was not previously known that the prostate can contribute any exosomes to the total urine exosome-pool. In healthy donors there was no positive staining for the prostate markers PSA or PSMA, and the tumour marker 5T4 was also negative. In the patient cohort, PSA was evident in 8/20, and PSMA present in 9/20 specimens (where 20/24 specimens were positive for one or more exosome-markers; i.e. evaluable as exosome-positive). Staining for 5T4 showed positivity in 14/20 samples. Together, this demonstrates for the first time, expression of prostate and cancer-associated markers by urinary exosomes.

One particular patient (p8) demonstrated the presence of comparable exosomes at each of the three time points, and a clear loss of exosomal-PSA in response to therapy, showing a strong band for PSA at ADT₄, which diminished with treatment, becoming undetectable at RT₂₀. Unexpectedly, however, 5T4 remained strongly expressed, even following 20-fractions of radiotherapy, suggesting this may be a candidate marker for assessing the presence of residual malignant cells, refractory to the effects of androgen-ablation or radiotherapy. The data are summarised in FIG. 6.

### Materials and Methods for Examples 1 to 5

### Prostate Cancer Patients and Healthy Donors

Ten PCa patients, participating in a local Phase II Clinical Trial, were recruited, together with 10 healthy male volunteers. The patients were confirmed positive for PCa by biopsy, and the tumour stage, Gleason score, serum-PSA and age is summarised in Table 2. Patients received 3-6 months neoadjuvant androgen deprivation therapy (ADT) prior to radical radiotherapy (RT), which consisted of a single phase delivering 55 Gy in 20 fractions to the prostate and 44 Gy in 20 fractions to the pelvic nodes. Patients were continued on adjuvant ADT according to clinical need. The trial was approved by the South East Wales Ethics Committee and informed consent was obtained from patients and volunteers participating in the study.

Details of urine specimens collected from healthy donors in given in Table 3.

TABLE 2
Details of patients participating in this study

| | | | | Serum | Serum | Serum |
|---|---|---|---|---|---|---|
| | Clinical | | | PSA | PSA | PSA at 6 |
| | Stage | Gleason | Age | ADT₄ | ADT₁₂ | months |
| Patient | (all N0) | Score | (years) | (ng/ml) | (ng/ml) | (ng/ml) |
| 1 | T2b | 7 (3 + 304) | 66 | 10.5 | 2.10 | 1.2 |
| 2 | T2b | 7 (3 + 304) | 62 | 134.0 | 0.20 | <0.01 |
| 3 | T2 | 8 (3 + 305) | 70 | 8.3 | 1.40 | <0.1 |
| 4† | n/d | 7 (3 + 304) | 65 | 83.2 | 83.40 | † |
| 5 | T2c | 7 (3 + 304) | 69 | 95.2 | 4.10 | <0.1 |
| 6 | T2 | 8 (4 + 304) | 70 | 10.8 | 0.10 | <0.1 |
| 7 | T3a | 7 (3 + 304) | 53 | 36.5 | 7.20 | 0.3 |
| 8 | T3b | 6 (3 + 303) | 61 | 14.1 | 0.80 | 0 |
| 9 | T2 | 7 (4 + 303) | 66 | 21.1 | 0.20 | <0.1 |
| 10 | T2 | 8 (4 + 304) | 71 | 28.1 | 1.3 | n/d |

†Patient died from an unrelated brain tumour prior to Radiation Treatment.
n/d not determined.

TABLE 3
Details of urine specimens collected from Healthy Donors

| | | Dip-Stick | | | | | Specimen | Exosomes | Exosome |
|---|---|---|---|---|---|---|---|---|---|
| Healthy | Age of | Blood, Protein, Glucose, | | | | | Volume | Recovered | Concentration |
| Donor | donor | Ketones, pH | | | | | (ml) | (μg) | (ng/ml) |
| 1 | 29 | 0 | 0 | 0 | 0 | 7 | 180 | 9.8 | 544 |
| 2 | 37 | 0 | 1 | 0 | 0 | 7 | 180 | 115.2 | 640.0 |
| 3 | 37 | 0 | 1 | 0 | 0 | 7 | 180 | 32.3 | 179.4 |
| 4 | 63 | 2 | 0 | 0 | 0 | 5 | 180 | 55.4 | 307.8 |
| 5 | 61 | 0 | 1 | 0 | 0 | 7 | 180 | 154.7 | 859.4 |
| 6 | 50 | 0 | 1 | 0 | 0 | 7 | 180 | 8.7 | 48.3 |
| 7 | 49 | 0 | 0 | 0 | 0 | 6 | 150 | 61.2 | 408.0 |
| 8 | 55 | 0 | 1 | 0 | 0 | 6 | 180 | 37.2 | 206.7 |
| 9 | 56 | 0 | 0 | 4 | 0 | 7 | 145 | 28.5 | 196.6 |
| 10 | 57 | 0 | 1 | 0 | 0 | 8 | 170 | 130.3 | 766.5 |

### Urine Sample Collection

Urine specimens, of up to 200 ml volume, were collected into sterile plastic containers (Millipore), and brought to the laboratory for processing within 30 minutes. Samples were collected mid to late morning, and these were not first-morning urine. Urine was tested for blood, proteins, glucose and Ketones and the pH was measured; (by Combur⁵ Test®D, dipstick (Roche)), the results are presented in Table 4. PCa-patient urine was collected at three time points: “ADT₄” (0-4 weeks after initiation of ADT), “ADT₁₂” (following three months of ADT) and “RT₂₀” (after 20 fractions of Radiotherapy). At intervals during treatment (ADT₄, ADT₁₂ and at 4 weeks post Radiotherapy), serum PSA levels were measured.

TABLE 4
Details of urine specimens collected from PCa patients

| | | Dip-Stick | | | | | Specimen | Total Exosomes | Exosome |
|---|---|---|---|---|---|---|---|---|---|
| | Time | Blood, Protein, Glucose, | | | | | Volume | Recovered | Concentration |
| Patient | Point | Ketones, pH | | | | | (ml) | (μg) | (ng/ml) |
| 1 | ADT₄ | 1 | 1 | 0 | 0 | 7 | 90 | 72.9 | 810.0 |
| | ADT₁₂ | 0 | 1 | 0 | 0 | 5 | 180 | 141.9 | 788.3 |
| | RT₂₀ | 0 | 0 | 0 | 0 | 7 | 180 | 19.6 | 109.3 |
| 2 | ADT₄ | 1 | 1 | 0 | 0 | — | 170 | 125.5 | 738.2 |
| | ADT₁₂ | 1 | 2 | 0 | 0 | 7 | 180 | 2.61 | 14.5 |
| | RT₂₀ | 0 | 0 | 0 | 0 | 5 | 90 | 39.2 | 435.7 |
| 3 | ADT₄ | 4 | 2 | 4 | 0 | 5 | 180 | 72.9 | 405.3 |
| | ADT₁₂ | 2 | 1 | 1 | 0 | 5 | 180 | 70.9 | 393.9 |
| | RT₂₀ | 0 | 1 | 1 | 0 | 5 | 60 | 8 | 133.3 |
| 4 † | ADT₄ | 0 | 1 | 0 | 0 | 5 | 95 | 25.4 | 268.0 |
| | ADT₁₂ | 1 | 3 | 3 | 0 | 5 | 55 | 6.54 | 118.9 |
| | RT₂₀ | — | — | — | — | — | — | — | — |
| 5 | ADT₄ | 4 | 0 | 0 | 0 | 5 | 180 | 38.4 | 213.6 |
| | ADT₁₂ | 1 | 2 | 0 | 0 | 7 | 90 | 27.1 | 301.2 |
| | RT₂₀ | 1 | 1 | 0 | 0 | 6 | 150 | 5.1 | 34.5 |
| 6 | ADT₄ | 0 | 0 | 0 | 0 | 6 | 180 | 19.4 | 108.1 |
| | ADT₁₂ | 1 | 0 | 0 | 0 | 5 | 180 | 6.2 | 34.7 |
| | RT₂₀ | 1 | 1 | 0 | 0 | 5 | 120 | 9.1 | 76.1 |
| 7 | ADT₄ | 3 | I | 1 | 0 | 6 | 97 | 39 | 402.1 |
| | ADT₁₂ | 0 | 1 | 0 | 0 | 5 | 120 | 12.1 | 101.0 |
| | RT₂₀ | 1 | 1 | 0 | 0 | 5 | 45 | 17.7 | 395.1 |
| 8 | ADT₄ | 0 | 1 | 1 | 0 | 6 | 150 | 125.1 | 834.4 |
| | ADT₁₂ | 0 | 1 | 0 | 0 | 5 | 110 | 26 | 236.4 |
| | RT₂₀ | 1 | | 0 | 0 | 7 | 60 | 34.4 | 574.0 |
| 9 | ADT₄ | 0 | 1 | 0 | 0 | 5 | 120 | 8.2 | 68.3 |
| | ADT₁₂ | 0 | 1 | 0 | 0 | 6 | 180 | 17 | 94.4 |
| | RT₂₀ | 2 | 3 | 4 | 0 | 6 | 60 | 133.1 | 2218.7 |
| 10 | ADT₄ | 0 | 1 | 0 | 0 | 5 | 120 | 19.4 | 162.3 |
| | ADT₁₂ | 0 | 0 | 0 | 0 | 7 | 180 | 11.4 | 63.4 |
| | RT₂₀ | 0 | 0 | 0 | 0 | 6 | 170 | 88.3 | 519.4 |

† Patient 4 died before RT
— Not recorded, or sample unavailable

### Exosome Purification

Fresh urine was subjected to serial centrifugation, removing cells (300 g, 10 minutes), removing non-cellular debris (e.g. casts, crystals, membrane fragments etc) (2000 g, 15 minutes, repeated until there was no visible pellet). The supernatant was then underlayed with a 30% sucrose/D2O cushion, and subjected to ultracentrifugation at 100,000 g for 2 hours (SW32 Rotor, Optima LE80K Ultracentrifuge, Beckman Coulter). The cushion was collected, diluted in at least 7× volume PBS, and exosomes pelleted by a further ultracentrifugation step at 100,000 g, 2 hours, using a 70Ti rotor (Beckman Coulter). Exosome pellets were resuspended in 100-150 ul of PBS and frozen at −80° C. The quantity of exosomes present in each pellet was determined by the micro BCA protein assay (Pierce/Thermo Scientific).

### Cell Culture

LNCaP and DU145 prostate cancer cell lines (from ATCC), were seeded into bioreactor flasks (from Integra), and maintained at high density culture for exosome production. The bioreactor flasks were fed every 7 days, with conditioned medium kept for exosome purification as above.

### Electrophoresis and Immuno-Blotting

Cell lysates were compared to exosomes by immuno-blotting. Briefly, equal quantities of protein (5 μg per well) were solubilised by addition of 30% volume of 6M Urea, 50 mM Tris-HCL, 2% SDS and 0.002% w/v bromophenol blue. Samples were electrophoresed through 10% polyacrylamide gels, and transferred to polyvinylidine difluoride (PVDF) membranes which were blocked overnight in 3% w/v non-fat milk, 0.05% v/v Tween-20 in PBS (PBS-T). Primary antibodies were incubated for 1 hour, following 5 washes in PBS-T, and goat-anti mouse-Ig-HRP conjugate, from Santa Cruz (at 1:35,000 dilution) was added for 30 minutes. After 5 washes in PBS-T, bands were detected using the ECL+ system (Amersham/GE healthcare). Primary monoclonal antibodies included mouse anti-human PSA (a gift from Dr Atilla Turkes, Cardiff and Vale NHS Trust, Cardiff), anti-TSG101, anti-LAMP-1, anti-HSP90, anti-Calnexin, anti-CD81 and anti-PSMA (from Santa Cruz Biotechnology), anti GAPDH (from BioChain Institute, Inc), anti CD9 (from R&D systems). Anti-5T4 was a gift from Dr R Harrop (Oxford BioMedica UK Ltd). Goat polyclonal anti-Tamm Horsfall Protein (THP) was from Santa Cruz, and bands were detected using anti-goat-HRP (Dako). Membranes were stripped using the Restore Plus™ western blotting stripping buffer (Pierce/Thermo Scientific), blocked overnight, and re-probed.

### Examining Exosome Membrane Integrity

To investigate if urine damages exosome-membranes, exosomes isolated from B-cell lines, were immobilised onto anti-MHC Class-II coated dynal-beads (Dynal/Invitrogen). The exosome-bead complexes incubated overnight at 37° C. in 25 mM Calcein-AM. Calcein-loaded exosome-bead complexes were exposed to various salt-solutions or to fresh urine, at room temperature for 1 h. Fluorescence was analysed by flow cytometry (FACScan, BD), running Cell Quest software (BD). Calcein-fluorescence was compared to fluorescence of anti-Class-I (RPE) stained exosome-beads, in parallel tubes; a measure of whether exosomes remain attached to the bead surface. Results are expressed as the ratio of Calcein:Class-I fluorescence.

### Examining Proteolytic Damage of Exosomes by Urine

Exosomes purified from LNCaP cells, were treated with fresh urine in the presence or absence of protease inhibitors (including EDTA, Pepstatin-A, Leupeptin and PMSF). After 2 hours or 18 hours, samples were examined by western blot for expression of CD9, PSA and TSG101. As a positive control for proteolysis, exosomes were treated with trypsin (Cambrex).

### Example 6

### Proteomic Analysis of Bladder Cancer Exosomes

### 6.1 Characterisation of HT1376 Exosomes

Cultured HT1376 cells (a typical and well characterised transitional cell carcinoma (TCC) line) were the source of exosomes for this study. The present inventors used a previously developed method utilising differential ultracentrifugation and flotation on a 30% Sucrose/D2O cushion, to isolate exosomes from cell conditioned medium (Lamparski et al (2002) I Immunol Methods 270, 211-226) and Andre (2002) Lancet 360, 295-305) that separates exosomes from non-exosomal material based upon the previously defined exosome flotation characteristics (Raposo et al (1996) as above). Exosomes purified in this manner were subjected to several forms of analysis to evaluate sample quality/purity prior to analysis using proteomic workflows. Firstly, western blots were performed to compare whole cell lysates with exosomes to examine the expression of expected exosomal markers (according to published reports describing other exosome types) (Théry et al (2006) Curr Protoc Cell Biol, UNIT 3.22) and to evaluate the relative expression of these markers compared to the parent cell as a whole. As expected, the multivesicular body marker TSG101 was strongly enriched in exosome preparations compared with cell lysates (FIG. 7A).

Additionally a number of other molecules, including MHC Class-I, the tetraspanins CD9, CD81, the lysosomal protein LAMP-1, and to some extent GAPDH were similarly enriched. Such features are typical of exosomes produced by varied cell types (Théry et al (2006) as above). The heat shock protein hsp90 was not exosomally enriched and this is typical of cells which are not under cell-stress conditions (Mitchell et al (2008) J. Immunol. Methods 335, 98-105, Clayton et al (2005) J Cell Sci 118. 3631-3638 and Dai et al (2005) Clin Cancer Res 11, 7554-7563). Staining for cytokeratin-18 revealed a strong band in cell lysates but little or no detectable band in exosomes. Similarly the endoplasmic reticulum resident gp96 was readily detected in cell lysates by not in exosomes which indicated that little if any contaminating cellular debris was present in the exosome preparations. The phenotype of the exosome surface was also examined following coupling to latex beads by flow cytometry (FIG. 7B). This was performed in order to demonstrate the expression of correctly oriented proteins at the exosome surface. Tetraspanins were the choice markers for this because their expression is a well documented feature of exosomes from multiple cell types. The analyses showed very strong expression of tetraspanins, of the order CD9>CD81>CD63 typical for this and other cancer cell lines (unpublished observations) (FIG. 7B). Moreover, this assay can also highlight the presence of significant contaminating protein in the preparations, where contaminants rather than exosomes would bind to the bead surface during the coupling reaction, and subsequently yield low fluorescence signal for exosomal markers like CD9 (FIG. 7B line graph). Intentional contamination of purified exosomes with FBS (the likeliest source of contaminants in our system) reveals that adding 0.01% FBS is sufficient to decrease CD9-specific staining by around 30%. Exosome preparations staining below 5000 median fluorescence units (for CD9 staining) were deemed low quality and not utilised further. As well as expression of a typical exosomal molecular profile, another key feature of exosomes was also investigated; that is their density characteristics. HT1376 exosomes, pelleted at 70,000×g were overlaid on a linear sucrose gradient and subjected to ultracentrifugation for 18 hours. Fifteen fractions were collected, and analysis by western blot revealed the presence of TSG101 floating at a density range around 1.1-1.19 g/ml (FIG. 7C). Such analysis demonstrates that HT1376 cells produce exosomes of typical density similar to that described for exosomes from other cell types (Raposo et al (1996) as above). This method, in combination with the latexmicrobead assay (above) was also used as a tool for validating MS protein identifications in the latter section of this manuscript. Electron microscopy of preparations was also performed (FIG. 7D) revealing nano-vesicular structures within a size range consistent for their definition as exosomes (30-100 nm). Taken together the data indicate that HT1376 bladder cancer cells produce exosomes that have molecular and biophysical characteristics similar to exosomes of other cell types, and that our exosome preparations from this source are of high quality, and are low/free of contaminating cellular debris.

### 8.2 Identification of Exosomal Proteins by Nano LC-MALDI TOF/TOF

In order to obtain exosome-derived trypsin-digest peptides for nano-LC, the present inventors modified version of the standard protocol encompassing a 1% (w/v) SDS extraction to solubilise membrane proteins. (Tan et al (2008) Proteomics 8, 3924-3932). This modified protocol involved pelletting previously-prepared HT1376 exosomes by ultracentrifugation, and boiling this pellet in a TEAB-buffer containing 1% (w/v) SDS and 20 mM DTT. The inclusion of DTT here was added to enhance solubilisation. An additional ultracentrifugation step was also included after this (spinning at 118,000 g/45 minutes) in order to remove any remaining insoluble material and/or aggregates. The supernatant was subjected to a solvent based precipitation method to remove SDS, salts and lipids (2D cleanup, GE Healthcare). The resulting pellet of exosome proteins was quantified by protein assay prior to trypsin digestion, and nano-LC. This process resulted in the identification of 353 proteins (2-peptides or more). The full set of identifications is listed in Table 6 and note that only high confidence protein identifications have been reported (two or more peptides with high quality MS/MS data). Due to these selection criteria our FDR was 0%. In common with many other proteomics laboratories the single peptide has not reported identifications although inevitably some of these assignments will be valid. Exploring the nano-LC/MS identifications revealed several proteins consistent with exosome biosynthesis. For example, members of the ubiquitin-dependent complex ESCRT (endosomal sorting complex required for transport), were present including vacuolar protein sorting-associated protein 28 homolog (vps-28) and, vacuolar protein sorting-associated protein 4B (vps-4B), ubiquitin-like modifier-activating enzyme and ubiquitin. These identifications suggest a multivesicular body origin for the sample analysed. Proteins involved in membrane trafficking and fusion processes were also evident (Clathrin heavy chain 1, Rab-11B, Rab-5A, Rab-6a, Rab-7a, Rab GDP dissociation inhibitor beta, Annexin A1, A2, A3, A4, A5, A6, A7, Annexin A8-like protein and Annexin A11). Markers of endosome/lysosome were also present (EH domain-containing protein 1 and 2, Lysosome membrane protein 2, Lysosome associated membrane protein-2, tripeptidyl-peptidase 1, Cathepsin-D, Sequestosome-1), and several proteins with chaperone functions were identified (hsp70, hsc70, hsp90, stress-induced-phosphoprotein 1, T-complex protein 1, endoplasmin) Components of the cytosol are also expected to be found within the exosome lumen, a natural consequence of the membrane budding process during multivesicular body formation, and here also a diverse assortment of cytosolic enzymes (Glyceraldehyde-3-phosphate dehydrogenase, cytosol aminopeptidase, cytosolic acetyl-CoA acetyltransferase, nicotinate phosphoribosyltransferase) was found and cytoskeletal constituents (actin, Alpha-actinin-4, cytokeratins, ezrin, tubulin, myosin). Diverse transmembrane proteins were also abundant, (including multiple integrins β1, β4, α3, α6, αv,) MHC molecules, CD9, EGF receptor, MUCIN-1, CD44, syndecan-1) and various membrane transporters (Solute carrier family 2 and 3, 4F2 cell-surface antigen heavy chain, Choline transporter-like protein, Sodium/potassium-14 transporting ATPase subunit beta-3). The proteome identified here is therefore broadly consistent with that expected for exosomes, being comparable to proteomic identifications highlighted by other researchers investigating exosomes from other cellular or physiological sources (FIG. 8A) (Simpson et al (2009) Expert Rev Proteomics 6, 267-283).

### 8.3 Exocarta and Gene Ontology Analysis

To assist interpretation of the entire protein identification set, a brief bioinformatic analysis of our MS data was undertaken, comparing the identifications firstly with the Exocarta database (a repository for previous proteomic studies of exosomes), and secondly identifying key biological themes in the data using GeneGO Metacore (Version 5.4) The Exocarta database collates lists of EntrezGene IDs that have been extracted from exosome research publications. Over-representation analysis (ORA) using the hypergeometic distribution was applied to explore whether there were more genes overlapping with Exocarta gene sets than could be expected by chance. The present inventors limited the comparisons to studies utilizing MS-based proteomics approaches, and to those with at least 10 matching (Wubbolts et al (2003) J Biol Chem 278, 10963-10972) identifications and FDR corrected the results to control for multiple testing. A summary plot of the findings is included (FIG. 8A). The results indicate this data set as being consistent with other studies of exosomes. Importantly, however, very significant data matching was seen when comparing our data with exosomes isolated from colorectal carcinoma cells. Proteins related to carcinoma, therefore, must feature heavily in the current data set; containing protein identifications capable of distinguishing neoplastic from non-neoplastic epithelia. A similar, unbiased overrepresentation analysis was also performed using both gene ontology and the proprietary curated gene sets within GeneGO Metacore, with queries made under four categories; disease biomarker, diseases in general, biological process and cellular compartment, respectively. For disease biomarker, our data indicated the most significant association to be with bladder cancer; supporting therefore the premise that exosome-analysis may well be a useful tool for disease-specific biomarker identification. Other biomarker associations included carcinomas of colon and breast (FIG. 8B). Similarly, a query examining general disease associations revealed features related to cancer of the gastrointestinal tract, metastatic cancer, respiratory tract diseases (including lung cancer) and carcinoma (FIG. 8C) It was surprising not to see urological tract related diseases featuring within the top ten (shown), albeit significant relationships with the genitourinary tract (including bladder neoplasm) were present in the top 40-significant associations. This ORA analysis reveals therefore that HT1376 exosomes express proteins strongly related to neoplastic diseases in general, and to carcinomas in particular (FIGS. 8B and 8C). Examining biological processes associated with this proteome revealed significant associations with the control of the cytoskeleton, intercellular adhesion, matrix adhesion processes, and protein folding related processes (FIG. 8D). In terms of cellular compartment, the proteome was strongly related to membranous vesicle within the cytoplasm, the cytoplasm and the cytoskeleton. The top associations however specified melanosome and pigment granule compartments (FIG. 8E). The nucleus, endoplasmic reticulum and mitochondria did not feature as significantly associated compartments. In conclusion, the statistically-based, unbiased analyses presented here, demonstrate aspects of bladder cancer exosome proteome that are similar to exosomes from other sources, but moreover, emphasise a proteome particularly implicated in carcinoma.

### 8.4 Validation of the Nano-LC Approach Using 2DE

The present inventors performed 2DE with the aim of selecting random spots for MS identification, and to confirm the absence/presence of these proteins in the main identification list (Table 6). Although such gels were attempted using around 100 μg of purified exosomes, the spot picks contained too little material to yield confident protein identifications, with <10% of attempts yielding successful identifications. Up scaling this process using around 500 μg of exosomes per gel, however, resulted in an identification hit rate of >53%. Seventeen spots of intermediate staining intensity (silver stained), were successfully identified by MS analysis. These included integrin α3 and α6, Gelsonin, cytosolic enzymes LDH and GAPDH, cytoskeleton proteins actin and cytokeratins, Ezrin and others. Nineteen of the 21 identifications from this gel based approach were also identified by the nano-LC method, demonstrating excellent agreement (90%) between these different methods for resolving exosomal proteins/peptides (Data are summarised in FIG. 10).

### 8.5 Validation of Proteins Identified: Anomalous MHC Class-I Identifications

As with any such proteomic data set, it is important to evaluate the list manually for any unexpected or unexplainable MS identifications, and to question the validity of any anomalies discovered in the data. In the current analysis, the nano-LC/MS data contained multiple identifications for 16 HLA molecules, which passed our quality criteria (Expect values <0.05, and ID's based on more than one peptide). These identifications, however, were not physiologically possible. These included five HLA-B alleles and five HLA-C alleles (Table 5). Explanations for this could include contamination of the source cell line with other cells from different donor(s), inadvertent contamination of the specimen by researchers, or issues related to how MASCOT designated HLA-haplotypes nomenclature based on the peptide sequences generated from MS. To address these possibilities, a clinical diagnostic service (Welsh Blood Service, Llantrisant, Wales, UK), was employed to haplotype the researcher and the HT1376 cell line The researcher had no HLA alleles that corresponded to those in the MS list, whilst HT1376 was haplotyped as HLA-A*24; B*15(62); Cw*03(9), and is therefore confirmed a homogenous cell line. This led us to examine in more detail the peptide sequences obtained, and to evaluate how these were assigned by MASCOT to a given HLA-nomenclature (Table 5). It was apparent that several peptide sequences had been assigned to multiple HLA-types. For example sequence FDSDAASPR was designated to HLA-B15, B52, B54, B59 and to HLA-001, C12, C17 and C03. In contrast, however, there were some peptides which appeared in only a single designation. These unique sequences were assigned to HLA-A24 (APWIEQEGPEYWDEETGK, AYLEGTCVDGLR and WEAAHVAEQQR), HLA-C03 (GEPHFIAVGYVDDTQFVR) and HLA-G (APWVEQEGPEYWEEETR, FIAMGYVDDTQFVR and THVTHHPVFDYEATLR). There were no unique peptides for any HLA-B allele, although of the HLA-B subtypes identified, HLA-B15 was assigned the greatest number of peptides. In conclusion, manual analysis of peptides designated as MHC Class-I identifications is recommended to clarify potential confusion arising from such MASCOT results.

### 8.6 Validation of Exosomal Expression of Proteins Identified

It is also important to determine the validity of some MS-identified proteins, by identifying their presence in the sample by other techniques. With a list as large as 353 proteins it was not possible to do this wholesale so the present inventors restricted such validation to a set of proteins that may be of biological interest. A series of western blot panels was performed, analysing up to 20 μg HT1376 exosomes per well, to determine whether some MS-identified proteins were detectable in our exosome preparations. The present inventors stained for TSG101 as our choice marker for multivesicular bodies (and hence exosomes), a protein that was incidentally detected by MS by only a single peptide sequence, and was excluded therefore from our data on this basis. Lysosomal associated membrane protein-2 (LAMP2), a molecule which was expected to be present in exosomes was detected in the sample by MS and was confirmed here to be strongly positive by western blot (FIG. 8A). Among the MS identifications were numerous cytokeratin identifications (type-I cytoskeletal keratins 1, 7, 13, 14, 16, 17, 18, 19). Although found in other exosomeproteomic studies, the loading of such cytoskeletal components into exosomes is an aspect that has not been particularly highlighted as a biological feature of exosomes. The present inventors have confirmed expression of cytokeratin 17 and cytokeratin 18 in the preparations, revealing abundant expression of exosomal cytokeratin 17. Cytokeratin 18, however, was only detectable with 20 μg exosomes per well, suggesting that exosomes genuinely do express multiple cytoskeletal constituents, and that the nano-LC/MS approach is sufficiently sensitive to detect molecules such as CK18 that are difficult to reveal by traditional western blot methods. Because of the anomalous issues surrounding MHC identifications, it was important determine whether or not HLA-G was in fact expressed by HT1376 exosomes, as this was not included in the PCR-haplotyping of HT1376 cells. HLA-G was confirmed positive unequivocally here by western blot. Other membrane associated (Galectin-3, Basigin and CD73) or soluble (hnRNPK, β-catenin) molecules with documented associations in varied aspects of cancer biology, are confirmed positively expressed by HT1376 exosomes. Although the standard exosome purification method used here is robust, it remains possible that some non exosomal contaminating material is present in the preparations, and that some of these MS identifications are not genuinely exosomally expressed proteins. To try and address this issue, linear sucrose gradient preparations were performed, from HT1376 cell conditioned medium, in order to determine the capacity of the identified proteins to float at exosomal densities. Each of 15 collected fractions was split ⅓rd for analysis by flow cytometry (of exosome-coated beads) and ⅔rd for western blotting. The former method would reveal possible expression of candidate proteins at the exosome surface, while solubilising exosomes for western blot would allow surface and intra-lumenal constituents to be revealed. In the flow cytometry assay, exosome-containing fractions were identified by strong staining for tetraspanins CD9 and CD81 and for MHC Class-I, known to be expressed on the surface of HT1376 exosomes, revealing a clear (and principal) peak at a density of 1.12 g/ml (FIG. 9B), which is within the expected exosomal density (FIG. 7C). This fraction, containing most of the exosomes therefore, also revealed positive surface staining for the MS-identified proteins β1 and α6 integrin, CD36, CD44, CD73, CD10, MUC1, trophoblast glycoprotein (5T4) and Basigin. The same fractions were also stained with a calnexin specific antibody, revealing low level expression predominantly at densities greater than the exosome containing fractions, highlighting the specificity of positive-staining for the other markers tested, and showing the absence of this protein in exosome containing fractions as expected (FIG. 8B). To reveal relevant fractions in the western blot panel, the present inventors stained for TSG101, highlighting densities of 1.12-1.2 g/ml as exosome-containing. There was some positive staining at hyperdense fractions (>1.2 g/ml), but this was relatively weak, and may be due to exosome or protein aggregates. The proteins 5T4, CD44, Basigin, Galectin-3 and β-catenin, all co-localised at the same density range, consistent with their exosomal expression. The data show that MS-identifications achieved in this study are expressed by HT1376-exosomes, and that membrane-associated molecules, often difficult to solubilise and identify by MS-approaches have been successfully identified and validated as localised to the exosome membrane.

In summary, the present inventors have achieved the first high quality proteomic description of bladder cancer cell-derived exosomes, using highly pure exosome preparations, rigorous specimen quality control, strict MS criterion and substantive validation. The information gathered using this system may ulitmately be used to replace the highly invasive procedures currently utilised in diagnosis and monitoring this disease, with a fully non-invasive urinary exosome-based technique.

### Materials and Methods for Example 6

Cell culture—HT1376, is a cell line originating from a primary transitional cell carcinoma (TCC) of the bladder (Stage T2, Grade G4) (Gardner et al (1997) J Natl Cancer Inst 58, 881-890). These cells were used as the exosome source for this study because they have been extensively characterised previously, and are well representative of the behaviour and phenotype of TCC (Gardner et al (1997) as above; and Masters et al 1986 Cancer Res 46, 3630-3636). The cells were maintained in DMEM, supplemented with pen/strep and 5% FBS (which had been depleted of exosomes by overnight ultracentrifugation at 100,000 g, followed by filtration through 0.2 μm and then 0.1 μm vacuum filters, Millipore). The cells were seeded into bioreactor flasks (from Integra), and maintained at high density culture for exosome production as described (Mitchell et al Immuno Methods 335, 98-105). Cells were confirmed negative for mycoplasma contamination by monthly screening (mycoalert, Lonza).

Exosome purification—Cell culture medium was subject to serial centrifugation, removing cells (300 g, 10 minutes), removing cellular debris (2000 g, 15 minutes). The supernatant was then centrifuged at 10,000 g for 30 minutes, and the supernatant was retained. This was underlaid with a 30% sucrose/D2O cushion, and subjected to ultracentrifugation at 100,000 g for 2 hours. The cushion was collected, and exosomes washed in PBS, as described (Lamparski et al (2002) Immunol Methods, 270, 211-226; Andre et al (2002) Lancet 360, 295-305; and Clayton et al (2007) Cancer Res 67, 7458-7466). Exosome pellets were resuspended in 100-150 ul of PBS and frozen at −80° C. The quantity of exosomes was determined by the micro BCA protein assay (Pierce/Thermo Scientific). Transmission electron microscopy of preparations was performed as described (Clayton et al (2007) as above).

Determination of exosome density—To quantify the density of exosomes produced by HT1376, a protocol similar to that previously described was used, based on ultracentrifugation on a linear sucrose gradient (Raposo et al (1996) J. Exp. Med. 183, 1161-1172 and Théry et al (2006) Curr Protoc Cell Biol, UNIT 3.22). Briefly, cell culture supernatant was subjected to differential centrifugation and the pellet at 70,000 g was overlaid on a linear sucrose gradient (0.2M up to 2.5M Sucrose). Specimens were centrifuged at 4° C., overnight at 210,000 g, using an MLS-50 rotor in an Optima-Max ultracentrifuge (Beckman Coulter). The refractive index of collected fractions was measured (at 20° C.) using an automatic refractometer (J57WR-SV, Rudolph Scientific), and from this, the density was calculated as described (Raposo et al (1996) as above). Fractions were washed in buffer (PBS or MES buffer; discussed below), by ultracentrifugation at 150,000 g (in a TLA-110 rotor, Optima-Max Ultracentrifuge), and pellets resuspended in MES-buffer for coupling to microbeads, or in SDS-sample buffer for analysis by western blot.

Flow cytometric analyses of exosome-coated beads—One microgram of purified exosomes was incubated with 1 μl of latex beads (surfactant free, aldehyde-sulphate 3.9 μm beads, Interfacial Dynamics, Oregon), that had been washed twice in IVIES buffer (0.025M MES, 0.154M NaCl, pH6). For analysis of sucrose gradient fractions, 30% of each fraction was coupled to 0.5 μl of stock beads. Exosome-beads were incubated in a final volume of 100 μl MES buffer at RT for 1 hour, on a shaking platform, thereafter rolling overnight at 4° C. Beads were blocked by incubating with 1% BSA/MES buffer, for 2 hours at RT. Blocking buffer was washed away, and beads resuspended in 0.1% BSA/MES buffer. Primary antibodies were added (at 2-10 μg/ml), for 1 hour at 4° C. After one wash, goat anti-mouse-Alexa-488 conjugated antibody in 0.1% BSA/MES buffer (at 1:200, Invitrogen) was added for 1 hour. After washing, beads were analysed by flow cytometry, using a FACSCanto instrument, configured with a high throughput sampling module, running FACSDiva v6.1.2 software (Becton Dickinson).

1D electrophoresis and immuno-blotting—Cell lysates were compared to exosome-lysates by immuno-blotting as described (Clayton et al (2003) Eur. J. Immunol. 33, 552-531), where protein (up to 20 μg per well) were solubilised by addition of 30% volume of 6M Urea, 50 mM Tris-HCL, 2% SDS, 20 mM DTT and 0.002% w/v bromophenol blue. Samples were electrophoresed through 4-12% Bis-Tris gels (Invitrogen), and transferred to polyvinylidine difluoride (PVDF) membranes which were blocked and probed with antibodies using the Qdot® system (Invitrogen). Bands were visualised using the MiniBIS Pro imaging system (DNR Bio-Imaging Systems). Primary monoclonal antibodies specific against TSG101, LAMP-1, HSP90, Calnexin, HLA-G, Galectin-3, Basigin, hnRNPK, cytoketatins 18 and 17 and CD44 were from Santa Cruz Biotechnology. Anti GAPDH (from BioChain Institute, Inc), anti CD9 (from R&D systems), and anti CD81 (from Serotec). Anti-5T4 was a gift from Dr R Harrop (Oxford BioMedica UK Ltd).

2D electrophoresis and MS—A gel based approach was employed to examine the exosome protein profile, using a standard 2DE protocol. Briefly exosomes (750 μg) were solubilised for 1 hour at room temperature in 150 μl of lysis buffer (7M urea, 2M thiourea, 20 mM DTT, 4% (w/v) CHAPS, 0.005% (w/v) bromophenol blue and 0.5% (v/v) immobilised pH gradient (IPG) buffer pH 3-10NL (GE Healthcare)). Extracted proteins were then solvent precipitated using the 2D-Clean Up kit (GE Healthcare) before the pellet was resuspended in lysis buffer. Isoelectric focussing of the sample was performed using 18 cm pH 3-10NL IPG rehydrated strips, an Ettan IPGphor III IEF system (GE Healthcare) and recommended voltages. Subsequently the IPG strip was equilibrated for 15 minutes in equilibration buffer (50 mM Tris-HCl pH8.8, 6M urea, 2% (w/v) SDS, 30% (v/v) glycerol, 0.002% (w/v) bromophenol blue) containing 1% (w/v) DTT followed by 15 minutes in equilibration buffer containing 2.5% (w/v) iodoacetamide. Equilibrated IPG strips were subject to second dimension separation using the Ettan™ DALTsix system (GE Healthcare). Silver staining was performed and randomly selected gel spots were excised, subjected to trypsin digestion and MALDI-TOF/TOF mass spectrometry analysis as previously described (Brennan et al (2009) Proteomics-Clin Apps 3, 359-369). The database search settings employed were the same as described for LC-MALDI protein identification except that a precursor mass tolerance of 50 ppm was used.

Preparation of exosome derived peptides for nano-LC—HT1376-derived exosome preparations were re-pelleted at 118,000 g for 45 minutes at 4° C. in a TLA-110 rotor, Optima-Max Ultracentrifuge (Beckman Coulter). The pellets were solubilised in 100 μl triethylammonium bicarbonate (TEAB) lysis buffer (20 mM TEAB) containing 20 mM DTT and 1% (w/v) SDS at RT for 10 minutes, followed by 95° C. for 10 minutes, and left for a further 10 minutes at RT. The samples were subject to an additional ultracentrifugation step (118,000 g for 45 minutes at RT) and supernatants (now free of insoluble material) were subjected to solvent precipitation to remove salts, lipids and detergent (using 2D clean-up, GE Healthcare). The pellets were resuspended in 20 mM TEAB and left overnight at 4° C. The protein content was then determined using a BCA protein assay kit (Sigma). Samples were then reduced, denatured and alkylated using an Applied Biosystems iTRAQ labelling kit and standard protocol. The proteins were subjected to digestion with trypsin, 0.8 μg per sample and incubated at 37° C. for 12 to 16 hours. The samples were then dried and resuspended in water with 0.1% (v/v) TFA.

LC-MALDI and protein identification—Digested peptides were separated on a nano-LC system (UltiMate 3000, Dionex, Sunnyvale, USA) using a two-dimensional salt plug method as previously described (Brennan et al (2009) Proteomics-Clin Apps 3, 359-369). Mass spectrometry was performed using an Applied Biosystems 4800 MALDI TOF/TOF mass spectrometer as described in Brennan et al (2009). The MS/MS data was used to search the Swiss-Prot database (Version 57.1; release date 14 Apr. 2009; 462764 sequences; human taxonomy) using the MASCOT Database search engine v2.1.04 (Matrix Science Ltd, London, UK) embedded into GPS Explorer software v3.6 Build 327 (Applied Biosystems) (default GPS parameters, 1 missed cleavage allowed, fixed modification of MMTS(C), variable modifications of oxidation (M), pyro-glu (N-term E) and pyro-glu (Nterm Q), 150 ppm mass tolerance in MS and 0.3 Da mass tolerance for MS/MS). In order for a protein to be identified there needed to be a minimum of two peptides with MASCOT evalues less than 0.05. There was a false discovery rate (FDR) of 0% which was determined using the same SwissProt database with the entire sequence randomised. The analysis was performed with two biological replicates, each including a technical replicate.

MS data analysis—The resultant protein list was analysed for any biological enrichment against defined lists using Metacore GeneGO (Version 5.4) and from selected ExoCarta submissions (MS based data containing 10 or more matching gene identifiers). For analysis using the Exocarta gene sets our protein list was converted from SwissProt Accession to EntezGene ID's using BioMart, before over-representation analysis (ORA) using the hypergeometic distribution in R against a background of all human genes with EntrezGene IDs. For ORA in MetaCore data was first converted into SwissProt IDs (using BioMart) before analysis, again using hypergeometic tests.

### Example 7

### Detection of 5T4 Expression by Exosomes by ELISA

The capture antibody CD9 ((clone 209306—IgG2b) purified mouse anti-human (carrier protein free) antibody (R&D Systems, US)) is diluted in dPBS (Lonza, UK) to a working concentration of 10 μg/ml and 100 μl added to each well to be used (1 μg/well). The capture antibody is incubated on the ELISA plate (96-well Flat-bottomed, F-stripped, High binding ELISA plate (Greiner bio9-oine Ltd, UK)) at 4° C. for 18 hours. The plate wells are then washed three times with 300 μl/well DELFIA™ (Perkin Elmer) wash solution.

In order to block non-specific binding the reagent diluent (10× concentrate 2 (10% BSA) (R&D Systems, US)) is diluted ten times in dPBS (10%) to produce 1% BSA. 300 μl is then added to each well to be blocked, and incubated for 2 hours at room temperature. The plate wells are then washed three times with 300 μl/well DELFIA wash solution.

To capture exosomes, 100-200 μl of a exosome-containing sample (such as a biological fluid or cell condition medium) is added to each well and incubated at room temperature for 2 hours. The plate wells are washed three times with 300 μ/well DELFIA wash solution.

Detection may be performed with a 5T4 antibody (5T4 (clone H8)—biotin conjugated antibody (Oxford BioMedica, Oxford UK)) by diluting the 5T4 antibody in DELFIA assay buffer to a working concentration of 0.1 μg/ml, adding 100 μl to each well for detection (0.01 μg or 10 ng/well) incubating at room temperature for 2 hours, and then washing the plate wells with 300 μl/well DELFIA wash solution.

A 5T4-biotin antibody may be Europium-streptavidin labelled by diluting the Europium-streptavidin in DELFIA assay buffer 1/1000, adding 100 μl to each well, incubating at room temperature for 45 minutes and washing the plate wells six times with 300 μl/well DELFIA wash solution.

The Europium signal is acquired by adding 100 μl of the DELFIA enhancement solution to each well, incubating at room temperature for 5 minutes while mixing and reading the plate on a Wallac Victor 2 Multi-label Counter plate reader (Perkin Elmer). The results are shown in FIG. 11.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in cellular studies using flow cytometry or related fields are intended to be within the scope of the following claims.

1. A method for detecting a 5T4-positive cancer in a subject comprising:
(i) identifying exosomes in and/or purifying exosomes from a sample from the subject; and
(ii) detecting exosome-associated 5T4.

2. The method according to claim 1, which comprises detecting a 5T4 peptide, polypeptide or nucleic acid encoding such a peptide or polypeptide.

3. The method according to claim 1, wherein the 5T4-positive cancer is a cancer of the genitourinary tract.

4. The method according to claim 3, wherein the cancer is prostate cancer.

5. The method according to claim 1, wherein the sample is a urine sample.

6. The method according to claim 1, wherein the sample is, or is derivable from, a blood sample.

7. The method according to claim 6, wherein the sample is a serum sample.

8. The method according to claim 1, that further comprises detecting of one or more:
(i) biomarkers for the cancer, in addition to 5T4;
(ii) exosomal markers; and/or
(iii) markers for a particular tissue or cell-type.

9. The method according to claim 8, wherein the one or more markers of the particular tissue are prostate markers.

10. The method according to claim 9, of wherein the one or more prostate markers comprise PSA and/or PSMA.

11. A method for determining whether a cancer is a 5T4-positive cancer, the method comprising:
(i) identifying exosomes in and/or purifying exosomes from a sample from a subject that has been diagnosed with cancer; and
(ii) detecting exosome-associated 5T4,
wherein the detection of 5T4 confirms that the cancer is 5T4-positive.

12. A method for treating cancer in a subject comprising:
(i) determining that the cancer in the subject is 5T4-positive by a method according to claim 11; and
(ii) administering a 5T4-based therapeutic to the subject.

13. A method of identifying a subject that will be suitable for treatment with a 5T4-based therapeutic, the method comprising detecting exosome-associated 5T4 in a sample from the subject, wherein the detection of the 5T4 in the sample identifies the subject as being suitable for the treatment.

14. A method for monitoring the progression of a 5T4-positive cancer, the method comprising measuring the level of exosome-associated 5T4 in a sample from the subject and comparing the levels of exosome-associated 5T4 in samples taken from a subject at a plurality of time points, wherein an increase in the level of exosome-associated 5T4 over time indicates worsening of the cancer, and a reduction in the level of exosome-associated 5T4 over time indicates amelioration of the cancer.

15. The method according to claim 14 wherein samples are taken from the subject at a plurality of time points during treatment.

16. The method according to claim 15, wherein the treatment comprises administration of a 5T4-based therapeutic.

17. A kit for detecting a 5T4-positive cancer in a subject, which comprises:
(i) an exosome detection, collection and/or purification system; and
(ii) a detection system for 5T4.

18. The kit according to claim 17, wherein the detection system detects 5T4 peptide, polypeptide or nucleic acid encoding such a peptide or polypeptide.

19. The kit according to claim 17, wherein the detection system also quantifies the level of 5T4.

20. A method according to claim 1, wherein 5T4 in the sample is detected using one or more of the following methods:
(i) binding to an anti-5T4 antibody;
(ii) binding to a T cell receptor specific for a 5T4 peptide;
(iii) binding to a nucleic acid sequence showing a high degree of identity to the complement of all or part of a nucleic acid sequence encoding 5T4; and
(iv) amplification of a 5T4 nucleic acid using 5T4-specific primers.

21. The method of claim 13, comprising administering a 5T4-based therapeutic to the subject.


SEQUENCE LISTING (48 sequences)

SEQ ID NO: 1 - protein, 9 aa, Homo sapiens
PLADLSPFA

SEQ ID NO: 2 - protein, 10 aa, Homo sapiens
LHLEDNALKV

SEQ ID NO: 3 - protein, 10 aa, Homo sapiens
LEDNALKVLH

SEQ ID NO: 4 - protein, 9 aa, Homo sapiens
HLEDNALKV

SEQ ID NO: 5 - protein, 9 aa, Homo sapiens
LEDNELKVL

SEQ ID NO: 6 - protein, 8 aa, Homo sapiens
LADNALKV

SEQ ID NO: 7 - protein, 21 aa, Homo sapiens
YRYEINADPRLTNLSSNSSDV

SEQ ID NO: 8 - protein, 20 aa, Homo sapiens
QTSYVFLGIVLALIGAIFLL

SEQ ID NO: 9 - protein, 18 aa, Homo sapiens
APWIEQEGPEYWDEETGK

SEQ ID NO: 10 - protein, 12 aa, Homo sapiens
AYLEGTCVDGLR

SEQ ID NO: 11 - protein, 11 aa, Homo sapiens
WEAAHVAEQQR

SEQ ID NO: 12 - protein, 18 aa, Homo sapiens
GEPHFIAVGYVDDTQFVR

SEQ ID NO: 13 - protein, 17 aa, Homo sapiens
APWVEQEGPEYWEEETR

SEQ ID NO: 14 - protein, 14 aa, Homo sapiens
FIAMGYVDDTQFVR

SEQ ID NO: 15 - protein, 16 aa, Homo sapiens
THVTHHPVFDYEATLR

SEQ ID NO: 16 - protein, 13 aa, Homo sapiens
QEYDESGPSIVHR

SEQ ID NO: 17 - protein, 16 aa, Homo sapiens
SYELPDGQVITIGNER

SEQ ID NO: 18 - protein, 15 aa, Homo sapiens
GAGTDEGCIEILASR

SEQ ID NO: 19 - protein, 16 aa, Homo sapiens
GLGTDEDAIISVLAYR

SEQ ID NO: 20 - protein, 11 aa, Homo sapiens
KAPDFVFYAPR

SEQ ID NO: 21 - protein, 15 aa, Homo sapiens
EVQGFESATFLGYFK

SEQ ID NO: 22 - protein, 14 aa, Homo sapiens
LVINGNPITIFQER

SEQ ID NO: 23 - protein, 17 aa, Homo sapiens
EAGNPGSLFGYSVALHR

SEQ ID NO: 24 - protein, 19 aa, Homo sapiens
NSYPDVAVGSLSDSVTIFR

SEQ ID NO: 25 - protein, 16 aa, Homo sapiens
GIVSKDEITFVSGAPR

SEQ ID NO: 26 - protein, 17 aa, Homo sapiens
DGEVGGAVYVYMNQQGR

SEQ ID NO: 27 - protein, 11 aa, Homo sapiens
DEITFVSGAPR

SEQ ID NO: 28 - protein, 11 aa, Homo sapiens
FYNELTEILVR

SEQ ID NO: 29 - protein, 12 aa, Homo sapiens
NPEQEPIPIVLR

SEQ ID NO: 30 - protein, 12 aa, Homo sapiens
AFHNEAQVNPER

SEQ ID NO: 31 - protein, 18 aa, Homo sapiens
VAPEEHPVLLTEAPLNPK

SEQ ID NO: 32 - protein, 9 aa, Homo sapiens
AEIDMLDIR

SEQ ID NO: 33 - protein, 16 aa, Homo sapiens
GAGTDEGCLIEILASR

SEQ ID NO: 34 - protein, 10 aa, Homo sapiens
APDFVFYAPR

SEQ ID NO: 35 - protein, 10 aa, Homo sapiens
HVVPNENWQR

SEQ ID NO: 36 - protein, 10 aa, Homo sapiens
AGAHLQGGAK

SEQ ID NO: 37 - protein, 9 aa, Homo sapiens
YLLLAGAPR

SEQ ID NO: 38 - protein, 13 aa, Homo sapiens
YTQVLWSGSEDQR

SEQ ID NO: 39 - protein, 23 aa, Homo sapiens
FLTALAQDGVINEEALSVTELDR

SEQ ID NO: 40 - protein, 18 aa, Homo sapiens
FLLDHQGELFPSPDPSGL

SEQ ID NO: 41 - protein, 10 aa, Homo sapiens
EQLAIAEFAR

SEQ ID NO: 42 - protein, 12 aa, Homo sapiens
NHLLHVFDEYKR

SEQ ID NO: 43 - protein, 8 aa, Homo sapiens
QLFDQVVK

SEQ ID NO: 44 - protein, 10 aa, Homo sapiens
ARPVINIVHK

SEQ ID NO: 45 - protein, 16 aa, Homo sapiens
DGWQDIVIGAPQYFDR

SEQ ID NO: 46 - protein, 8 aa, Homo sapiens
ELPELLQR

SEQ ID NO: 47 - protein, 8 aa, Homo sapiens
LEQLGIPR

SEQ ID NO: 48 - protein, 9 aa, Homo sapiens
FDSDAASPR